(19) United States
(12) Patent Application Publication
(10) Pub. No.: US 2010/0209463 A1
(43) Pub. Date: Aug. 19, 2010

(54) UV FILTER CAPSULE

(75) Inventors: Frank Pfluecker, Darmstadt (DE); Bernd Mueller, Zwingenberg (DE); Gabriele Witte, Buettelborn (DE); Valerie Andre, Ludwigshafen (FR)

Correspondence Address: MILLEN, WHITE, ZELANO & BRANIGAN, P.C., 2200 CLARENDON BLVD., SUITE 1400, ARLINGTON, VA, 22201, US

(73) Assignees: MERCK PATENT GMBH, Darmstadt (DE); BASF SE, Ludwigshafen (DE)

(21) Appl. No.: 12/670,442

(22) PCT Filed: Jul. 2, 2008
(86) PCT No.: PCT/EP08/05408

§ 371 Date: Jan. 25, 2010

(30) Foreign Application Priority Data
Jul. 26, 2007 (DE) 10 2007 035 567.1

(51) Int. Cl. A61K 8/02; A61Q 17/04

(52) U.S. Cl. 424/401; 424/59

(57) ABSTRACT

The present invention relates to UV filter capsules, to the use thereof for the preparation of cosmetic or dermatological formulations or dispersions, and to cosmetic or dermatological formulations which comprise the capsules, and to a process for the production thereof.

The present invention relates to UV filter capsules, to the use thereof for the preparation of cosmetic or dermatological formulations or dispersions, and to cosmetic or dermatological formulations which comprise the capsules, and to a process for the preparation thereof.

It is generally known that the ultraviolet part of sunlight has a harmful effect on the skin. While rays having a wavelength smaller than 290 nm (so-called UVC region) are absorbed by the ozone layer in the earth's atmosphere, rays in the range between 290 nm and 320 nm, the so-called UVB region, cause erythema, simple sunburn or even various degrees of burning.

It has also been shown that rays in the range between about 320 nm and 400 nm (UVA region) result in damage to the elastic and collagenic fibres of the connective tissue, which causes the skin to age prematurely. Furthermore, these rays are the cause of numerous phototoxic and photoallergic reactions. The harmful influence of UVB radiation may be augmented by UVA radiation.

However, UV radiation can also result in photochemical reactions, with the photochemical reaction products then engaging in the skin metabolism. Furthermore, UV radiation counts amongst ionising radiation. There is thus a risk of UV exposure also being accompanied by the formation of ionic species, which are then themselves capable of engaging oxidatively in the biochemical processes.

The light-protection filters usual today in cosmetics and dermatology are therefore also divided into UVA and UVB filters. Numerous compounds are known for protection against UVB radiation, mostly derivatives of 3-benzylidenecamphor (for example Eusolex® 6300), of 4-aminobenzoic acid, of cinnamic acid, of salicylic acid, of benzophenone, of triazine and also of 2-phenylbenzimidazole. For protection against UVA radiation, use is frequently made of dibenzoylmethane derivatives, such as, for example, 4-(tert-butyl)-4′-methoxydibenzoylmethane (Eusolex® 9020) or 4-isopropyldibenzoylmethane (Eusolex® 8020), but these do not have unlimited stability on UV irradiation.

In view of the comments made above, the provision of cosmetic products having improved, more effective or optimised protection of the human skin against the damaging effects of UV-A and UV-B radiation is of major importance. It is particularly desirable here to provide compositions which achieve a desired action using the smallest possible amount of the individual components. The specific absorbance of the light-protection agents likewise plays a role, as does the stability of the emulsions prepared therewith, the toxicological acceptability thereof and the solubility thereof in the vehicles used (for example cosmetic oils). Some of the light-protection agents used to date are distinguished by good absorbance properties, but the low solubility of these substances prevents optimum use thereof. Thus, the UV filter substance Uvinul®T150 (INCI: Ethylhexyl Triazone), sold by BASF Aktiengesellschaft, is distinguished by excellent UV absorption properties, but this UV filter can only be dissolved in cosmetic oils to a limited extent and can only be incorporated into a number of formulations in relatively low proportions, which limits the protection factor that can be achieved by this filter.

There therefore continues to be a demand for improved administration forms of low-solubility organic UV filters which allow the use or application of these UV filters in larger amount. A water-soluble or dispersed administration form does not exist to date for Ethylhexyl Triazone.

It has now been found that low-solubility organic UV filters can be employed very well in encapsulated form if the capsule contains an emollient which is capable of dissolving the low-solubility organic UV filter to the extent of greater than 40% by weight at 25° C.

The present invention therefore relates firstly to UV filter capsules comprising

a polymeric shell and
a) at least one low-solubility organic UV filter and
b) an emollient which is capable of dissolving more than 40% by weight of the low-solubility organic UV filter at room temperature (20° C. to 25° C.).

Suitable capsules here can have walls of inorganic or organic polymers. For example, U.S. Pat. No. 6,242,099 B1 describes the production of suitable capsules having walls or a shell made from chitin, chitin derivatives or polyhydroxylated polyamines. Capsules which can particularly preferably be employed in accordance with the invention have walls or a shell which can be obtained by a sol-gel process, as described in the applications WO 00/09652, WO 00/72806, WO 00/71084, WO 03/39510 and WO 03/066209. Preference is in turn given here to capsules whose walls or shell are or is built up from silica gel (silica; undefined silicon oxide hydroxide). The production of corresponding capsules is known to the person skilled in the art, for example, from the patent applications cited, the relevant content of which expressly also belongs to the subject-matter of the present application. Capsules preferably used accordingly consist of a shell and a core.

The process preferably used for the production of the UV filter capsules according to the invention takes place in three steps:

in step a), an oil-in-water emulsion is prepared from a mixture comprising a sol-gel precursor for the production of the polymeric shell, at least one low-solubility UV filter and an emollient which is capable of dissolving more than 40% by weight of the low-solubility organic UV filter at room temperature (20° C. to 25° C.), in an aqueous solution,
in step b), the emulsion prepared in step a) is mixed to give an aqueous solution having a pH of 2 to 4, preferably of 3 to 4, and optionally in step c), reaction products are separated off from the sol-gel precursor.

The aqueous solution from step b) serves primarily for acceleration of the basic condensation-polymerisation reaction which causes build-up of the shell.

After an appropriate reaction time, in which the mixture can also be warmed or cooled or the pH can also be modified, the capsules formed can be isolated by means which are familiar to the person skilled in the art. For example, they can be centrifuged or filtered. A particularly preferred type of isolation is spray drying. This means that in step c), besides the separation of the reaction products from the sol-gel precursor, the UV filter capsules can, if necessary, be isolated.

In general, a dispersion or suspension comprising the UV filter capsules according to the invention in a form as can be employed directly in cosmetic or dermatological compositions is obtained after step c). Re-suspension of the isolated capsules in, for example, deionised water or in another medium is also conceivable and can be used for use in the compositions according to the invention.

The hydrophobic solution from step a) and also the aqueous solutions from steps a) and b) may comprise surfactants and/or other additives which may improve and/or stabilise this process and/or the product.

The sol-gel precursor used can be a metal or semi-metal alkoxide monomer, a metal ester, semi-metal ester or a partially hydrolysed and partially condensed polymer, or a mixture thereof.

Suitable and preferred sol-gel precursors are compounds of the formula M(R)ₙ(P)ₘ, in which M denotes a metal or semi-metal, preferably Si, R denotes a hydrolysable substituent, and n denotes an integer from 2 to 4, P denotes an unpolymerisable substituent, and m denotes an integer from 0 to 4, or a partially hydrolysed or partially condensed polymer thereof, or any mixture thereof.

The process described above is particularly preferably carried out using tetraethyl orthosilicate or a partially hydrolysed or partially condensed polymer thereof, or a mixture thereof. Tetraethyl orthosilicate is very particularly preferably employed as sol-gel precursor.

Further details are disclosed in the working examples.

For the purposes of the present invention, “low-solubility organic UV filters” are organic UV filters having a solubility in Dicaprylyl Carbonate (trade name Cetiol® CC, Cognis) of less than 25%, 26%, 27%, 28% or 30%, preferably less than 31%, 32%, 33%, 34% or 35%, particularly preferably less than 36%, 37%, 38%, 39% or 40% at room temperature (20° C. to 25° C.) and an experiment duration of 24 hours.

In a preferred embodiment, the at least one low-solubility organic UV filter mentioned under a) is a triazine derivative, diarylbutadiene derivative, hydroxybenzophenone derivative and/or methylenebisbenzotriazolyltetramethylbutylphenol derivative.

In the case of the triazine derivatives, the compounds 2,4,6-tris[anilino(p-carbo-2′-ethyl-1′-hexyloxy)]-1,3,5-triazine, dioctylbutamidotriazone, bisethylhexyloxyphenolmethoxyphenyltriazine, 2,4,6-tris(diethyl-4′-aminobenzal malonate)-s-triazine, 2,4,6-tris(dimethyl-4′-aminobenzal malonate)-s-triazine, 2,4,6-tris(diisopropyl-4-aminobenzal malonate)-s-triazine, 2,4,6-tris[3′-benzotriazol-2-yl)-2′-hydroxy-5′-methyl)phenylamino]-s-triazine and 2,4,6-tris[3′-benzotriazol-2-yl)-2′-hydroxy-5′-tert-octyl)phenylamino]-s-triazine are preferred. Particular preference is given here to the triazine derivatives 2,4,6-tris[anilino(p-carbo-2′-ethyl-1′-hexyloxy)]-1,3,5-triazine (Uvinul®T150, BASF Aktiengesellschaft, Ethylhexyl Triazone according to INCI), dioctylbutamidotriazone (UV-Sorb-HEB®, 3V Sigma) and bisethylhexyloxyphenol methoxyphenyltriazine (anisotriazine or Tinosorb®S, Ciba-Geigy). Very particular preference is given to the compounds 2,4,6-tris-[anilino-(p-carbo-2′-ethyl-1′-hexyloxy)]-1,3,5-triazine (Uvinul®T150, BASF Aktiengesellschaft) and/or dioctylbutamidotriazone (UV-Sorb-HEB®, 3V Sigma).

Further triazine derivatives are revealed by the patent applications P-A 0796851, EP-A 0087098 and EP-A 0850935.

In the case of the diarylbutadiene derivatives, the 4,4′-diarylbutadienes of the formula II

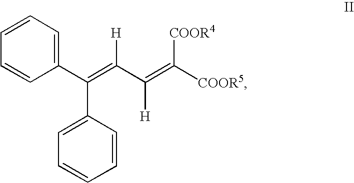

where R⁴ and R⁵, independently of one another, denote H, C₁-C₁₀-alkyl, C₃-C₁₀-cycloalkyl or C₃-C₁₀-cycloalkenyl, are preferred. Particular preference is given to the compound 1,1-dicarboxy(2′,2′-dimethylpropyl)-4,4-diphenylbutadiene. The 4,4′-diarylbutadienes mentioned are known as such, and their structure and preparation are described in the patent applications EP 0967200 and EP 916 335, the contents of which are expressly incorporated herein by way of reference.

In the case of the hydroxybenzophenones, the compounds of the general formula III

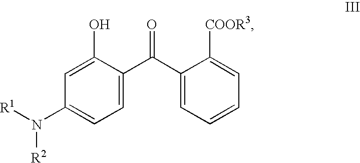

where R¹ and R², independently of one another, denote H, C₁-C₂₀-alkyl, C₃-C₁₀-cycloalkyl or C₃-C₁₀-cycloalkenyl, where the substituents R¹ and R², together with the nitrogen atom to which they are bonded, may form a 5- or 6-membered ring, and R³ denotes C₁-C₂₀-alkyl, are preferred. Particular preference is given to hexyl 2-(4-N,N-diethylamino-2-hydroxybenzoyl)benzoate (Uvinul®A Plus, BASF Aktiengesellschaft). Further examples of hydroxybenzophenones and the preparation thereof are revealed by the German patent application DE-A 11917906, the contents of which are expressly incorporated herein by way of reference.

Alkyl radicals which may be mentioned for substituents of the formulae II or III are branched or unbranched C₁-C₂₀-alkyl chains, preferably methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, n-octyl, 2-ethylhexyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl or n-eicosyl.

Alkenyl radicals which may be mentioned for substituents of the formula II or III are branched or unbranched C₂-C₁₀-alkenyl chains, preferably vinyl, propenyl, isopropenyl, 1-butenyl, 2-butenyl, 1-pentenyl, 2-pentenyl, 2-methyl-1-butenyl, 2-methyl-2-butenyl, 3-methyl-1-butenyl, 1-hexenyl, 2-hexenyl, 1-heptenyl, 2-heptenyl, 1-octenyl or 2-octenyl.

Cycloalkyl radicals which may be mentioned for substituents of the formula II or III are preferably branched or unbranched C₃-C₁₀-cycloalkyl chains, such as, for example, cyclopropyl, cyclobutyl, cyclopentyl, cyclohexyl, cycloheptyl, 1-methylcyclopropyl, 1-ethylcyclopropyl, 1-propylcyclopropyl, 1-butylcyclopropyl, 1-pentylcyclopropyl, 1-methyl-1-butylcyclopropyl, 1,2-dimethylcyclopropyl, 1-methyl-2-ethylcyclopropyl, cyclooctyl, cyclononyl or cyclodecyl.

Cycloalkenyl radicals which may be mentioned for substituents of the formula II or III are preferably branched or unbranched, C₃-C₁₀-cycloalkenyl chains having one or more double bonds, such as cyclopropenyl, cyclobutenyl, cyclopentenyl, cyclopentadienyl, cyclohexenyl, 1,3-cyclohexadienyl, 1,4-cyclohexadienyl, cycloheptenyl, cycloheptatrienyl, cyclooctenyl, 1,5-cyclooctadienyl, cyclooctatetraenyl, cyclononenyl or cyclodecyl.

In the case of the methylenebisbenzotriazolyltetramethylbutylphenol derivatives, 2,2′-methylenebis[6-(2H-benzotriazol-2-yl)-4-(1,1,3,3-tetramethylbutyl)phenol] (Tinosorb®M, Ciba-Geigy) is preferred. These compounds are also described in FR 2440933, the contents of which are expressly incorporated herein by way of reference.

In a preferred embodiment, the UV filter capsules according to the invention contain mixtures of the low-solubility UV filters mentioned above.

In a further preferred embodiment, the UV filter capsules according to the invention contain, as emollient, compounds of the formula I

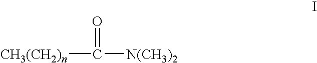

where n corresponds to an integer from 2 to 12.

The number “n” preferably stands for an integer from 3 to 11, preferably 4 to 10, particularly preferably 5 to 9, most preferably 6 to 8. In a preferred embodiment, the compound of the general formula I is N,N-dimethyldecanamide (CAS No. 14433-76-2), very particularly preferably Spectrasolv DMDA (Jiangsu Feixang Chemicals).

Spectrasolv DMDA is, in particular, an excellent emollient for Ethylhexyl Triazone, since the solubility thereof at room temperature (20° C. to 25° C.) is about 55% by weight. Owing to this excellent solubility, UV filter capsules whose content of Ethylhexyl Triazone is significantly different from the contents achieved for the good solvents for Ethylhexyl Triazone which are currently on the market have successfully been produced for the first time. A list in this respect is given in the working examples.

The UV filter capsules according to the invention here usually contain 1 to 90% by weight, preferably 5 to 70% by weight and particularly preferably 40 to 60% by weight of the at least one low-solubility organic UV filter, preferably in the core, i.e. surrounded by the polymeric shell. The core of the UV filter capsules according to the invention preferably consists of 1 to 90% by weight, preferably 5 to 70% by weight and particularly preferably 40 to 60% by weight of the at least one low-solubility organic UV filter, where the remainder of the capsule contents usually consists in accordance with the invention of the emollient which is capable of dissolving more than 40% by weight of the low-solubility organic UV filter at room temperature (20° C. to 25° C.) and optionally further assistants which further increase or stabilise the solubility of the organic UV filter.

The capsule contents of the capsules according to the invention, i.e. the core, therefore preferably consist of the at least one organic low-solubility UV filter and the emollient, mixtures of organic low-solubility UV filters, as described above, and the emollient or generally mixtures of at least one low-solubility organic UV filter and soluble organic UV filters in the emollient which is capable of dissolving the low-solubility organic UV filter to the extent of greater than 40% by weight at room temperature (20° C. to 25° C.).

Further preferred combinations are disclosed in the Claims.

The core very particularly preferably consists of Ethylhexyl Triazone and N,N-dimethyldecanimide.

The UV filter capsules according to the invention are furthermore characterised in that the core or capsule contents of the UV filter capsule comprises or consists of the at least one low-solubility UV filter mentioned under a) and the emollient mentioned under b) in the weight percent ratio 10:90 to 90:10, preferably in the weight percent ratio 30:70 to 70:30, particularly preferably in the weight percent ratio of 40:60 to 60:40 or very particularly preferably in the weight percent ratio of 50:50.

In detail, the encapsulation gives rise to the following advantages:
The hydrophilicity of the capsule wall (or synonymously thereto of the polymeric shell) can be adjusted independently of the solubility of the UV filter. The low-solubility organic UV filters can therefore be incorporated into purely aqueous formulations. This enables the low-solubility organic UV filter to be incorporated into both the oil phase and also the water phase of the cosmetic or dermatological composition as end product. The total content in cosmetic formulations can thus be increased.
If a low-solubility UV filter is present in the organic phase and the low-solubility UV filter encapsulated in accordance with the invention is present in the aqueous phase, a so-called boost effect is observed. In particular, a synergy occurs or in other words a boost if the same low-solubility UV filter is present in the organic phase and is present in encapsulated form in the water phase, which can be documented by in vivo SPF values of formulations without low-solubility UV filters encapsulated in accordance with the invention and with them.
The literature repeatedly discusses the penetration of skin by organic UV filters and the associated potential for irritation in the case of direct application to the human skin. The encapsulation of the corresponding substances proposed here suppresses this effect.
In general, encapsulation of individual UV filters or other ingredients enables formulation problems arising due to the interaction of individual formulation constituents with one another, such as crystallisation processes, precipitations and agglomeration, to be prevented since the interaction is suppressed.
The specific choice of the particular emollient, in particular the choice of N,N-dimethyldecanamide (Spectrasolv DMDA) enables the solubility of the at least one low-solubility organic UV filter, as described above, in particular of Ethylhexyl Triazone, to remain constant during the encapsulation, the UV filter not to crystallise and high loading of the capsule thus to be facilitated. The system described is furthermore temperature-stable, so that a stable capsule forms even during the possible temperature variations during production.
The low-solubility UV filter encapsulated in accordance with the invention, which can be employed in the water phase of a composition, can advantageously replace the usual aqueous UV filters in the water phase, such as, for example, phenylbenzimidazolesulfonic acid, since the requisite pH regulation for achieving stable compositions is unnecessary here.

The proportion of the UV filter capsules according to the invention in a dispersion is, as described above, preferably 5 to 80% by weight, particularly preferably 30 to 70% by weight, very particularly preferably 35 to 45% by weight, based on the total amount of the dispersion. In the working examples, the water content of the dispersions described is indicated as about 60% by weight.

The invention furthermore relates to the use of the UV filter capsules according to the invention, as described above, or of a dispersion comprising the UV filter capsules, as described above and below, for the preparation of a composition, in particular a cosmetic or dermatological composition. The invention furthermore relates to a dispersion or suspension comprising the UV filter capsules according to the invention, as described above. This dispersion is preferably an aqueous dispersion, i.e. the dispersion medium is water. Further dispersion media can also be any desired other suitable substances. Polyhydric alcohols, for example glycerol or 1,2-propanediol, are particularly suitable. The dispersion medium can also be a suitable mixture, for example a glycerol/water mixture in any desired ratio.

These are pre-dispersions, which are on the one hand themselves directly suitable as cosmetic or dermatological composition and on the other hand are able to simplify the preparation of such compositions which comprise a vehicle. The vehicle is preferably suitable for topical purposes, i.e. suitable for a local form which can, in particular, be applied to the surface.

The pre-dispersions according to the invention can be incorporated into the water phase of a composition, in particular into a cosmetic or dermatological composition.

The invention furthermore accordingly relates to a composition comprising at least one low-solubility organic UV filter and at least one suitable vehicle, in particular a vehicle which is suitable for topical purposes, characterised in that at least some of the low-solubility organic UV filter is encapsulated, more precisely in encapsulated form as described for the UV filter capsules according to the invention.

In order to achieve improved synergies with respect to the light protection factor, particular preference is given to compositions according to the invention which comprise an oil phase and an aqueous phase, where the UV filter capsules according to the invention are present in the aqueous phase, and at the same time at least one further oil-soluble UV filter or the low-solubility UV filter which is already located in the capsules, in particular the low-solubility UV filter, such as, for example, 2,4,6-tris[anilino-(p-carbo-2′-ethyl-1′-hexyloxy)]-1,3,5-triazine, hexyl 2-(4-diethylamino-2-hydroxy-benzoyl)benzoate, 1,1-dicarboxy-(2′,2′-dimethylpropyl)-4,4-diphenylbutadiene or bis-ethylhexyloxyphenolmethoxyphenyltriazine, is present in the oil phase.

The composition can include or comprise, essentially consist of or consist of the said necessary or optional constituents or ingredients. All compounds or components which can be used in the compositions are either known and commercially available or can be synthesised by processes which are known or described here.

For the purposes of the invention, the term composition is used synonymously with the term formulation or agent.

Skin-cosmetic, hair-cosmetic, dermatological, hygienic or pharmaceutical agents, compositions and/or formulations for topical application to skin or hair are suitable (i) for the prevention of damage to human skin and/or human hair, (ii) for the treatment of damage which has already occurred to human skin and/or human hair, (iii) for the care of human skin and/or human hair, (iv) for improving the skin feel (sensory properties). Agents for decorative cosmetics are explicitly included. Also included are agents for skin care, in which the pharmaceutical-dermatological application is achieved taking into account cosmetic points of view. Agents or compositions of this type are employed for the support, prevention and treatment of skin diseases and can develop a biological action in addition to the cosmetic effect. The compositions according to the invention are particularly preferably compositions for protection of the skin against damage by sunlight, especially by UV-B (280 to 320 nm) and UV-A radiation (>320 nm). These comprise, in a cosmetically tolerated medium, suitable assistants and additives, which are selected in consideration of the specific area of application. Assistants and additives of this type are familiar to the person skilled in the art and are revealed, for example, by cosmetics hand-books, for example Schrader, Grundlagen and Rezepturen der Kosmetika [Basic Principles and Formulations of Cosmetics], Hüthig Verlag, Heidelberg, 1989, ISBN 3-7785-1491-1, or Umbach, Kosmetik: Entwicklung, Herstellung and Anwendung kosmetischer Mittel [Cosmetics: Development, Preparation and Use of Cosmetic Compositions], 2nd expanded edition, 1995, Georg Thieme Verlag, ISBN 3 13 712602 9.

There are therefore in accordance with the invention, for example, the following processes for the preparation of compositions, in particular cosmetic or dermatological compositions, comprising capsules according to the invention:
A process for the preparation of a composition, in particular a cosmetic or dermatological composition having light-protection properties, is characterised in that the capsules according to the invention are mixed with further ingredients.
A process for the preparation of a composition, in particular a cosmetic or dermatological composition having light-protection properties, is characterised in that a pre-dispersion comprising the capsules according to the invention is mixed with further ingredients.
A particular process for the preparation of a composition, in particular a cosmetic or dermatological composition, in the form of an oil-in-water emulsion is characterised in that the pre-dispersion described above is emulsified with an oil.

The capsules here are preferably present in formulations according to the invention in amounts which ensure that the encapsulated UV filters are present in effective amounts in the formulation.

Preferably 1 to 40% by weight, particularly preferably 5 to 30% by weight, very particularly preferably 5 to 20% by weight, based on the total amount of the composition, of the UV filter capsules according to the invention are used in the compositions.

The size of the UV filter capsules according to the invention can vary from 0.001 to 20.0 μm, where the average particle size d(0.50), determined by a particle size determination by means of laser diffraction in accordance with ISO/DIS 13320, is generally 200 to 5000 nm, preferably 400 to 1500 nm. The method is described in the practical part.

The compositions, in particular the cosmetic or dermatological compositions, preferably having light-protection properties, can exist in various forms. Thus, it can be, for example, a solution, an emulsion or microemulsion of the water-in-oil (W/O) type or of the oil-in-water (O/W) type, a multiple emulsion, for example of the water-in-oil-in-water (W/O/W) type, a gel, a solid stick, an ointment or also an aerosol.

It is particularly preferred here for the cosmetic or dermatological composition to be an aqueous composition, in particular a gel, or an emulsion, in particular an oil-in-water emulsion (O/W emulsion), since the advantages of the formulations according to the invention come to bear particularly well in the preparation of such compositions.

The present invention therefore furthermore relates to emulsions comprising the formulation according to the invention described above in or as the water phase. Particular preference is given here to oil-in-water emulsions (O/W emulsions).

Emulsions according to the invention are advantageous and comprise, for example, the said fats, oils, waxes and other fatty substances, as well as water and an emulsifier, as usually used for a composition of this type.

The lipid phase may advantageously be selected from the following group of substances:
mineral oils, mineral waxes;
oils, such as triglycerides of capric or caprylic acid, furthermore natural oils, such as, for example, castor oil;
fats, waxes and other natural and synthetic fatty substances, preferably esters of fatty acids with alcohols having a low carbon number, for example with isopropanol, propylene glycol or glycerol, or esters of fatty alcohols with alkanoic acids having a low carbon number or with fatty acids;
silicone oils, such as dimethylpolysiloxanes, diethylpolysiloxanes, diphenylpolysiloxanes and mixed forms thereof.

For the purposes of the present invention, the oil phase of the emulsions, oleogels or hydrodispersions or lipodispersions is advantageously selected from the group of the esters of saturated and/or unsaturated, branched and/or unbranched alkanecarboxylic acids having a chain length of 3 to 30 C atoms and saturated and/or unsaturated, branched and/or unbranched alcohols having a chain length of 3 to 30 C atoms, from the group of the esters of aromatic carboxylic acids and saturated and/or unsaturated, branched and/or unbranched alcohols having a chain length of 3 to 30 C atoms. Ester oils of this type can then advantageously be selected from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of esters of this type, for example jojoba oil.

The oil phase may furthermore advantageously be selected from the group of the branched and unbranched hydrocarbons and waxes, silicone oils, dialkyl ethers, the group of the saturated or unsaturated, branched or unbranched alcohols, and fatty acid triglycerides, specifically the triglycerol esters of saturated and/or unsaturated, branched and/or unbranched alkanecarboxylic acids having a chain length of 8 to 24 C atoms, in particular 12-18 C atoms. The fatty acid triglycerides may advantageously be selected, for example, from the group of the synthetic, semi-synthetic and natural oils, for example olive oil, sunflower oil, soya oil, peanut oil, rapeseed oil, almond oil, palm oil, coconut oil, palm kernel oil and the like.

Any desired mixtures of oil and wax components of this type may also advantageously be employed for the purposes of the present invention. It may also be advantageous to employ waxes, for example cetyl palmitate, as the only lipid component of the oil phase.

The oil phase is advantageously selected from the group 2-ethylhexyl isostearate, octyldodecanol, isotridecyl isononanoate, isoeicosene, 2-ethylhexyl cocoate, C₁₂₋₁₅-alkyl benzoate, caprylic/capric acid triglyceride, dicapryl ether.

Particularly advantageous are mixtures of C₁₂₋₁₅-alkyl benzoate and 2-ethylhexyl isostearate, mixtures of C₁₂₋₁₅-alkyl benzoate and isotridecyl isononanoate, as well as mixtures of C₁₂₋₁₅-alkyl benzoate, 2-ethylhexyl isostearate and isotridecyl isononanoate.

Of the hydrocarbons, paraffin oil, squalane and squalene may advantageously be used for the purposes of the present invention.

Furthermore, the oil phase may also advantageously have a content of cyclic or linear silicone oils or consist entirely of oils of this type, although it is preferred to use an additional content of other oil-phase components in addition to the silicone oil or the silicone oils.

The silicone oil to be used in accordance with the invention is advantageously cyclomethicone (octamethylcyclotetrasiloxane). However, it is also advantageous for the purposes of the present invention to use other silicone oils, for example hexamethylcyclotrisiloxane, polydimethylsiloxane, poly(methylphenylsiloxane).

Also particularly advantageous are mixtures of cyclomethicone and isotridecyl isononanoate and of cyclomethicone and 2-ethylhexyl isostearate.

The aqueous phase of the compositions according to the invention optionally advantageously comprises alcohols, diols or polyols having a low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, furthermore alcohols having a low carbon number, for example ethanol, isopropanol, 1,2-propanediol, glycerol, and, in particular, one or more thickeners, which may advantageously be selected from the group silicon dioxide, aluminium silicates, polysaccharides and derivatives thereof, for example hyaluronic acid, xanthan gum, hydroxypropylmethylcellulose, particularly advantageously from the group of the polyacrylates, preferably a polyacrylate from the group of the so-called Carbopols, for example Carbopol grades 980, 981, 1382, 2984, 5984, in each case individually or in combination.

In particular, mixtures of the above-mentioned solvents are used. In the case of alcoholic solvents, water may be a further constituent.

Emulsions according to the invention are advantageous and comprise, for example, the said fats, oils, waxes and other fatty substances, as well as water and an emulsifier, as usually used for a formulation of this type.

Emulsifiers that can be used are, for example, the known W/O and O/W emulsifiers. It is advantageous to use further conventional co-emulsifiers in the preferred O/W emulsions according to the invention.

Co-emulsifiers which are advantageous in accordance with the invention are, for example, O/W emulsifiers, principally from the group of the substances having HLB values of 11-16, very particularly advantageously having HLB values of 14.5-15.5, so long as the O/W emulsifiers have saturated radicals R and R′. If the O/W emulsifiers have unsaturated radicals R and/or R′ or in the case of isoalkyl derivatives, the preferred HLB value of such emulsifiers may also be lower or higher.

It is advantageous to select the fatty alcohol ethoxylates from the group of the ethoxylated stearyl alcohols, cetyl alcohols, cetylstearyl alcohols (cetearyl alcohols). Particular preference is given to the following: polyethylene glycol (13) stearyl ether (steareth-13), polyethylene glycol (14) stearyl ether (steareth-14), polyethylene glycol (15) stearyl ether (steareth-15), polyethylene glycol (16) stearyl ether (steareth-16), polyethylene glycol (17) stearyl ether (steareth-17), polyethylene glycol (18) stearyl ether (steareth-18), polyethylene glycol (19) stearyl ether (steareth-19), polyethylene glycol (20) stearyl ether (steareth-20), polyethylene glycol (12) isostearyl ether (isosteareth-12), polyethylene glycol (13) isostearyl ether (isosteareth-13), polyethylene glycol (14) isostearyl ether (isosteareth-14), polyethylene glycol (15) isostearyl ether (isosteareth-15), polyethylene glycol (16) isostearyl ether (isosteareth-16), polyethylene glycol (17) isostearyl ether (isosteareth-17), polyethylene glycol (18) isostearyl ether (isosteareth-18), polyethylene glycol (19) isostearyl ether (isosteareth-19), polyethylene glycol (20) isostearyl ether (isosteareth-20), polyethylene glycol (13) cetyl ether (ceteth-13), polyethylene glycol (14) cetyl ether (ceteth-14), polyethylene glycol (15) cetyl ether (ceteth-15), polyethylene glycol (16) cetyl ether (ceteth-16), polyethylene glycol (17) cetyl ether (ceteth-17), polyethylene glycol (18) cetyl ether (ceteth-18), polyethylene glycol (19) cetyl ether (ceteth-19), polyethylene glycol (20) cetyl ether (ceteth-20), polyethylene glycol (13) isocetyl ether (isoceteth-13), polyethylene glycol (14) isocetyl ether (isoceteth-14), polyethylene glycol (15) isocetyl ether (isoceteth-15), polyethylene glycol (16) isocetyl ether (isoceteth-16), polyethylene glycol (17) isocetyl ether (isoceteth-17), polyethylene glycol (18) isocetyl ether (isoceteth-18), polyethylene glycol (19) isocetyl ether (isoceteth-19), polyethylene glycol (20) isocetyl ether (isoceteth-20), polyethylene glycol (12) oleyl ether (oleth-12), polyethylene glycol (13) oleyl ether (oleth-13), polyethylene glycol (14) oleyl ether (oleth-14), polyethylene glycol (15) oleyl ether (oleth-15), polyethylene glycol (12) lauryl ether (laureth-12), polyethylene glycol (12) isolauryl ether (isolaureth-12), polyethylene glycol (13) cetylstearyl ether (ceteareth-13), polyethylene glycol (14) cetylstearyl ether (ceteareth-14), polyethylene glycol (15) cetylstearyl ether (ceteareth-15), polyethylene glycol (16) cetylstearyl ether (ceteareth-16), polyethylene glycol (17) cetylstearyl ether (ceteareth-17), polyethylene glycol (18) cetylstearyl ether (ceteareth-18), polyethylene glycol (19) cetylstearyl ether (ceteareth-19), polyethylene glycol (20) cetylstearyl ether (ceteareth-20).

It is furthermore advantageous to select the fatty acid ethoxylates from the following group:

polyethylene glycol (20) stearate, polyethylene glycol (21) stearate, polyethylene glycol (22) stearate, polyethylene glycol (23) stearate, polyethylene glycol (24) stearate, polyethylene glycol (25) stearate, polyethylene glycol (12) isostearate, polyethylene glycol (13) isostearate, polyethylene glycol (14) isostearate, polyethylene glycol (15) isostearate, polyethylene glycol (16) isostearate, polyethylene glycol (17) isostearate, polyethylene glycol (18) isostearate, polyethylene glycol (19) isostearate, polyethylene glycol (20) isostearate, polyethylene glycol (21) isostearate, polyethylene glycol (22) isostearate, polyethylene glycol (23) isostearate, polyethylene glycol (24) isostearate, polyethylene glycol (25) isostearate, polyethylene glycol (12) oleate, polyethylene glycol (13) oleate, polyethylene glycol (14) oleate, polyethylene glycol (15) oleate, polyethylene glycol (16) oleate, polyethylene glycol (17) oleate, polyethylene glycol (18) oleate, polyethylene glycol (19) oleate, polyethylene glycol (20) oleate.

An ethoxylated alkyl ether carboxylic acid or salt thereof which can advantageously be used is sodium laureth-11 carboxylate. An alkyl ether sulfate which can advantageously be used is sodium laureth-14 sulfate. An ethoxylated cholesterol derivative which can advantageously be used is polyethylene glycol (30) cholesteryl ether. Polyethylene glycol (25) soyasterol has also proven successful. Ethoxylated triglycerides which can advantageously be used are the polyethylene glycol (60) evening primrose glycerides.

It is furthermore advantageous to select the polyethylene glycol glycerol fatty acid esters from the group polyethylene glycol (20) glyceryl laurate, polyethylene glycol (21) glyceryl laurate, polyethylene glycol (22) glyceryl laurate, polyethylene glycol (23) glyceryl laurate, polyethylene glycol (6) glyceryl caprate/caprinate, polyethylene glycol (20) glyceryl oleate, polyethylene glycol (20) glyceryl isostearate, polyethylene glycol (18) glyceryl oleate/cocoate.

It is likewise favourable to select the sorbitan esters from the group c polyethylene glycol (20) sorbitan monolaurate, polyethylene glycol (20) sorbitan monostearate, polyethylene glycol (20) sorbitan monoisostearate, polyethylene glycol (20) sorbitan monopalmitate, polyethylene glycol (20) sorbitan monooleate.

The following can be employed as optional W/O emulsifiers, but ones which may nevertheless be advantageous in accordance with the invention:

fatty alcohols having 8 to 30 C atoms, monoglycerol esters of saturated and/or unsaturated, branched and/or unbranched alkanecarboxylic acids having a chain length of 8 to 24 C atoms, in particular 12-18 C atoms, diglycerol esters of saturated and/or unsaturated, branched and/or unbranched alkanecarboxylic acids having a chain length of from 8 to 24 C atoms, in particular 12-18 C atoms, monoglycerol ethers of saturated and/or unsaturated, branched and/or unbranched alcohols having a chain length of 8 to 24 C atoms, in particular 12-18 C atoms, diglycerol ethers of saturated and/or unsaturated, branched and/or unbranched alcohols having a chain length of 8 to 24 C atoms, in particular 12-18 C atoms, propylene glycol esters of saturated and/or unsaturated, branched and/or unbranched alkanecarboxylic acids having a chain length of 8 to 24 C atoms, in particular 12-18 C atoms, and sorbitan esters of saturated and/or unsaturated, branched and/or unbranched alkanecarboxylic acids having a chain length of 8 to 24 C atoms, in particular 12-18 C atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol (2) stearyl ether (steareth-2), glyceryl monolaurate, glyceryl monocaprinate, glyceryl monocaprylate or PEG 30 dipolyhydroxystearate.

Cosmetic and dermatological compositions to be prepared in accordance with the invention additionally advantageously, although not necessarily, comprise inorganic pigments based on metal oxides and/or other metal compounds which are sparingly soluble or insoluble in water, in particular the oxides of titanium (TiO₂), zinc (ZnO), iron (for example Fe₂O₃), zirconium (ZrO₂), silicon (SiO₂), manganese (for example MnO), aluminium (Al₂O₃), cerium (for example Ce₂O₃), mixed oxides of the corresponding metals and mixtures of such oxides. Particular preference is given to pigments based on TiO₂ and in particular micronised TiO₂.

In accordance with the invention, the cosmetic and/or dermatological light-protection formulations can have the usual composition and serve for cosmetic and/or dermatological light protection, furthermore for the treatment, care and cleansing of the skin and/or of the hair and as make-up product in decorative cosmetics.

Particular preference is given in accordance with the invention to the preparation of cosmetic and dermatological compositions which are in the form of a sunscreen. These may advantageously additionally comprise at least one further UVA filter and/or at least one further UVB filter and/or at least one inorganic pigment, preferably hydrophobic inorganic micropigments.

Particular preference is given to UV filters whose physiological acceptability has already been demonstrated. For both UVA and UVB filters, there are substances which are known from the specialist literature, for example

benzylidenecamphor derivatives, such as 3-(4′-methylbenzylidene)dl-camphor (for example Eusolex® 6300), 3-benzylidenecamphor (for example Mexoryl® SD), polymers of N-{(2 and 4)-[(2-oxoborn-3-ylidene)methyl]benzyl}acrylamide (for example Mexoryl® SW), N,N,N-trimethyl-4-(2-oxoborn-3-ylidenemethyl)anilinium methylsulfate (for example Mexoryl® SK) or (2-oxoborn-3-ylidene)toluene-4-sulfonic acid (for example Mexoryl® SL),
benzoyl- or dibenzoylmethanes, such as 1-(4-tert-butylphenyl)-3-(4-meth-oxyphenyl)propane-1,3-dione (for example Eusolex® 9020) or 4-isopropyldibenzoylmethane (for example Eusolex® 8020),
benzophenones, such as 2-hydroxy-4-methoxybenzophenone (for example Eusolex® 4360) or 2-hydroxy-4-methoxybenzophenone-5-sulfonic acid and the sodium salt thereof (for example Uvinul® MS-40),
methoxycinnamic acid esters, such as octyl methoxycinnamate (for example Eusolex® 2292) or isopentyl 4-methoxycinnamate, for example as a mixture of the isomers (for example Neo Heliopan® E 1000),
salicylate derivatives, such as 2-ethylhexyl salicylate (for example Eusolex®OS), 4-isopropylbenzyl salicylate (for example Megasol®) or 3,3,5-trimethylcyclohexyl salicylate (for example Eusolex® HMS),
4-aminobenzoic acid and derivatives, such as 4-aminobenzoic acid, 2-ethylhexyl 4-(dimethylamino)benzoate (for example Eusolex® 6007) or ethoxylated ethyl 4-aminobenzoate (for example Uvinul® P25),
phenylbenzimidazolesulfonic acids, such as 2-phenylbenzimidazole-5-sulfonic acid and the potassium, sodium and triethanolamine salts thereof (for example Eusolex® 232), 2,2-(1,4-phenylene)bisbenzimidazole-4,6-disulfonic acid or salts thereof (for example Neoheliopan® AP) or 2,2-(1,4-phenylene)bisbenzimidazole-6-sulfonic acid;
and further substances, such as
2-ethylhexyl 2-cyano-3,3-diphenylacrylate (for example Eusolex® OCR),
3,3′-(1,4-phenylenedimethylene)bis(7,7-dimethyl-2-oxobicyclo[2.2.1]hept-1-ylmethanesulfonic acid and salts thereof (for example Mexoryl® SX) and
2,4,6-trianilino-(p-carbo-2′-ethylhexyl-1′-oxy)-1,3,5-triazine (for example Uvinul® T 150)
hexyl 2-(4-diethylamino-2-hydroxybenzoyl)benzoate (for example Uvinul®UVA Plus, BASF).

The compounds mentioned in the list should only be regarded as examples. It is of course also possible to use other UV filters:

These organic UV filters are generally incorporated into cosmetic formulations in an amount of 0.5 to 10 percent by weight, preferably 1-8% by weight.

Further suitable organic UV filters are, for example,
2-(2H-benzotriazol-2-yl)-4-methyl-6-(2-methyl-3-(1,3,3,3-tetramethyl-1-(trimethylsilyloxy)disiloxanyl)propyl)phenol (for example Silatrizole®),
2-ethylhexyl 4,4′-[(6-[4-((1,1-dimethylethyl)aminocarbonyl)phenylamino]-1,3,5-triazine-2,4-diyl)diimino]bis(benzoate) (for example Uvasorb® HEB),
dimethicone diethylbenzal malonate (CAS No. 207 574-74-1) or
2,2′-methylenebis(6-(2H-benzotriazol-2-yl)-4-(1,1,3,3-tetramethylbutyl)-phenol) (CAS No. 103 597-45-1)
2,2′-(1,4-phenylene)bis(1H-benzimidazole-4,6-disulfonic acid, monosodium salt) (CAS No. 180 898-37-7) and
2,4-bis{[4-(2-ethylhexyloxy)-2-hydroxy]phenyl}-6-(4-methoxyphenyl)-1,3,5-triazine (CAS No. 103 597-45-, 187 393-00-6).

Further suitable UV filters are also methoxyflavones corresponding to German patent application DE 10232595.

Organic UV filters are generally incorporated into cosmetic formulations in an amount of from 0.5 to 20 percent by weight, preferably 1-15%.

Conceivable inorganic UV filters are those from the group of the titanium dioxides, such as, for example, Coated titanium dioxide (for example Eusolex® T-2000, Eusolex® T-AQUA, Eusolex® T-AVO), zinc oxides (for example Sachtotec®), iron oxides or also cerium oxides. These inorganic UV filters are generally incorporated into cosmetic compositions in an amount of 0.5 to 20 percent by weight, preferably 2-10% by weight.

Preferred compounds having UV-filtering properties are 3-(4′-methylbenzylidene)dl-camphor, 1-(4-tert-butylphenyl)-3-(4-methoxyphenyl)propane-1,3-dione, 4-isopropyldibenzoylmethane, 2-hydroxy-4-methoxybenzophenone, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate, and the potassium, sodium and triethanolamine salts thereof.

Besides the compounds described here, the compositions according to the invention may also comprise at least one photostabiliser, preferably conforming to the formula I

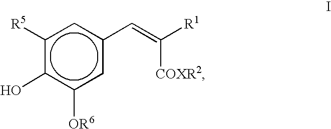

where
R¹ is selected from —C(O)CH₃, —CO₂R³, —C(O)NH₂ and —C(O)N(R⁴)₂;
X is O or NH,
R² is a linear or branched C₁₋₃₀-alkyl radical;
R³ is a linear or branched C₁₋₂₀-alkyl radical,
all R⁴, independently of one another, are H or linear or branched C₁₋₈-alkyl radicals,
R⁵ is H, a linear or branched C₁₋₈-alkyl radical or a linear or branched —O—C₁₋₈-alkyl radical, and
R⁶ is a C₁₋₈-alkyl radical,
   where the photostabiliser is particularly preferably bis(2-ethylhexyl) 2-(4-hydroxy-3,5-dimethoxybenzylidene)malonate. Corresponding photostabilisers and their preparation and use are described in International patent application WO 03/007906, the disclosure content of which expressly also belongs to the subject-matter of the present application. The compounds mentioned are also antioxidants.

It is furthermore possible and advantageous to combine the compositions according to the invention with antioxidants. A combination of this type then exhibits both a protective action as antioxidant and also against burns due to UV radiation. A protective action against oxidative stress or against the action of free radicals can thus also be achieved.

There are many proven substances known from the specialist literature which can be used as antioxidants, for example amino acids (for example glycine, histidine, tyrosine, tryptophan) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (for example buthionine sulfoximines, homocysteine sulfoximine, buthionine sulfones, penta-, hexa- and heptathionine sulfoximine) in very low tolerated doses (for example pmol to μmol/kg), and also (metal) chelating agents (for example α-hydroxy fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof, vitamin C and derivatives (for example ascorbyl palmitate, magnesium ascorbyl phosphate, ascorbyl acetate), tocopherols and derivatives (for example vitamin E acetate), vitamin A and derivatives (for example vitamin A palmitate), and coniferyl benzoate of benzoin resin, rutinic acid and derivatives thereof, α-glycosyl rutin, ferulic acid, furfurylideneglucitol, carnosine, butylhydroxytoluene, butylhydroxyanisole, nordihydroguaiaretic acid, trihydroxybutyrophenone, quercetin, uric acid and derivatives thereof, mannose and derivatives thereof, zinc and derivatives thereof (for example ZnO, ZnSO₄), selenium and derivatives thereof (for example selenomethionine), stilbenes and derivatives thereof (for example stilbene oxide, trans-stilbene oxide).

Suitable antioxidants are also compounds of the general formulae A or B

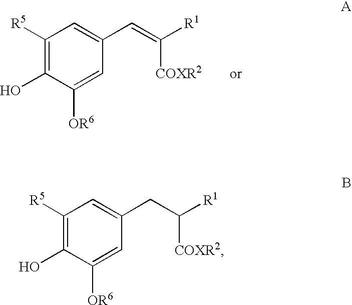

in which
R¹ can be selected from the group —C(O)CH₃, —CO₂R³, —C(O)NH₂ and —C(O)N(R⁴)₂,
X denotes O or NH,
R² denotes linear or branched alkyl having 1 to 30 C atoms,
R³ denotes linear or branched alkyl having 1 to 20 C atoms,
R⁴ in each case, independently of one another, denotes H or linear or branched alkyl having 1 to 8 C atoms,
R⁵ denotes linear or branched alkyl having 1 to 8 C atoms or linear or branched alkoxy having 1 to 8 C atoms and
R⁶ denotes linear or branched alkyl having 1 to 8 C atoms,
preferably derivatives of 2-(4-hydroxy-3,5-dimethoxybenzylidene)malonic acid and/or 2-(4-hydroxy-3,5-dimethoxybenzyl)malonic acid, particularly preferably bis(2-ethylhexyl) 2-(4-hydroxy-3,5-dimethoxybenzylidene)malonate (for example Oxynex® ST Liquid) and/or bis(2-ethylhexyl) 2-(4-hydroxy-3,5-dimethoxybenzyl)malonate (for example RonaCare® AP).

Mixtures of antioxidanants are likewise suitable for use in the cosmetic compositions according to the invention. Known and commercial mixtures are, for example, mixtures comprising, as active ingredients, lecithin, L-(+)-ascorbyl palmitate and citric acid (for example Oxynex® AP), natural tocopherols, L-(+)-ascorbyl palmitate, L-(+)-ascorbic acid and citric acid (for example Oxynex® K LIQUID), tocopherol extracts from natural sources, L-(+)-ascorbyl palmitate, L-(+)-ascorbic acid and citric acid (for example Oxynex® L LIQUID), DL-α-tocopherol, L-(+)-ascorbyl palmitate, citric acid and lecithin (for example Oxynex® LM) or butylhydroxytoluene (BHT), L-(+)-ascorbyl palmitate and citric acid (for example Oxynex® 2004).

The compositions according to the invention may comprise vitamins as further ingredients. The cosmetic compositions according to the invention preferably comprise vitamins and vitamin derivatives selected from vitamin A, vitamin A propionate, vitamin A palmitate, vitamin A acetate, retinol, vitamin B, thiamine chloride hydrochloride (vitamin B₁), riboflavin (vitamin B₂), nicotinamide, vitamin C (ascorbic acid), vitamin D, ergocalciferol (vitamin D₂), vitamin E, DL-α-tocopherol, tocopherol E acetate, tocopherol hydrogensuccinate, vitamin K₁, esculin (vitamin P active ingredient), thiamine (vitamin B₁), nicotinic acid (niacin), pyridoxine, pyridoxal, pyridoxamine (vitamin B₆), pantothenic acid, biotin, folic acid and cobalamine (vitamin B₁₂), particularly preferably vitamin A palmitate, vitamin C and derivatives thereof, DL-α-tocopherol, tocopherol E acetate, nicotinic acid, pantothenic acid and biotin.

The compositions according to the invention may in addition comprise further conventional skin-protecting or skin-care active ingredients. These may in principle be any active ingredients known to the person skilled in the art, such as, in particular, flavone derivatives, chromone derivatives, compatible solutes and other active ingredients.

It may be preferred for the composition according to the invention to comprise at least one repellent, where the repellent is preferably selected from N,N-diethyl-3-methylbenzamide, ethyl 3-(acetylbutylamino)propionate, dimethyl phthalate, butopyronoxyl, 2,3,4,5-bis(2-butylene)tetrahydro-2-furaldehyde, N,N-diethylcaprylamide, N,N-diethylbenzamide, o-chloro-N,N-diethylbenzamide, dimethyl carbate, di-n-propyl isocinchomeronate, 2-ethylhexane-1,3-diol, N-octylbicycloheptenedicarboximide, piperonyl butoxide, 1-(2-methylpropoxycarbonyl)-2-(hydroxyethyl)piperidine, or mixtures thereof, where it is particularly preferably selected from N,N-diethyl-3-methylbenzamide, ethyl 3-(acetylbutylamino)propionate, 1-(2-methylpropoxycarbonyl)-2-(hydroxyethyl)piperidine, or mixtures thereof.

The compositions according to the invention which comprise repellents are preferably insect repellents. Insect repellents are available in the form of solutions, gels, sticks, rollers, pump sprays and aerosol sprays, with solutions and sprays forming the majority of the commercially available products. The basis for these two product forms is usually formed by alcoholic or aqueous/alcoholic solutions with addition of fatting substances and slight perfuming.

In accordance with the invention, flavone derivatives is taken to mean flavonoids and coumaranones. In accordance with the invention, flavonoids is taken to mean the glycosides of flavonones, flavones, 3-hydroxyflavones (=flavonols), aurones, isoflavones and rotenoids [Römpp Chemie Lexikon [Römpp's Lexicon of Chemistry], Volume 9, 1993]. For the purposes of the present invention, however, this term is also taken to mean the aglycones, i.e. the sugar-free constituents, and the derivatives of the flavonoids and aglycones. For the purposes of the present invention, the term flavonoid is furthermore also taken to mean anthocyanidine (cyanidine). For the purposes of the present invention, the term coumaranones is also taken to mean derivatives thereof.

Preferred flavonoids are derived from flavonones, flavones, 3-hydroxyflavones, aurones and isoflavones, in particular from flavonones, flavones, 3-hydroxyflavones and aurones.

The flavonoids are preferably selected from the following compounds: 4,6,3′,4′-tetrahydroxyaurone, quercetin, rutin, isoquercetin, eriodictyol, taxifolin, luteolin, trishydroxyethylquercetin (troxequercetin), trishydroxyethylrutin (troxerutin), trishydroxyethylisoquercetin (troxeisoquercetin), trishydroxyethyl luteolin (troxeluteolin), α-glycosylrutin, tiliroside and the sulfates and phosphates thereof. Of the flavonoids, particular preference is given, as active substances according to the invention, to rutin, tiliroside, α-glycosylrutin and troxerutin.

Of the phenols having an antioxidative action, the polyphenols, some of which are naturally occurring, are of particular interest for applications for applications in the pharmaceutical, cosmetic or nutrition sector. For example, the flavonoids or bioflavonoids, which are principally known as plant dyes, frequently have an antioxidant potential. K. Lemanska, H. Szymusiak, B. Tyrakowska, R. Zielinski, I. M. C. M. Rietjens; Current Topics in Biophysics 2000, 24(2), 101-108, are concerned with effects of the substitution pattern of mono- and dihydroxyflavones. It is observed therein that dihydroxyflavones containing an OH group adjacent to the keto function or OH groups in the 3′,4′-or 6,7- or 7,8-position have antioxidative properties, while other mono- and dihydroxyflavones in some cases do not have antioxidative properties.

Quercetin (cyanidanol, cyanidenolon 1522, meletin, sophoretin, ericin, 3,3′,4′,5,7-pentahydroxyflavone) is frequently mentioned as a particularly effective antioxidant (for example C. A. Rice-Evans, N. J. Miller, G. Paganga, Trends in Plant Science 1997, 2(4), 152-159). K. Lemanska, H. Szymusials, B. Tyrakowska, R. Zielinski, A. E. M. F. Soffers, I. M. C. M. Rietjens; Free Radical Biology & Medicine 2001, 31(7), 869-881, have investigated the pH dependence of the antioxidant action of hydroxyflavones. Quercetin exhibits the greatest activity amongst the structures investigated over the entire pH range.

Suitable antioxidants are furthermore compounds of the formula II

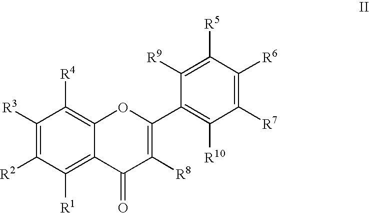

where R¹ to R¹⁰ may be identical or different and are selected from
H
OR¹¹
straight-chain or branched C₁- to C₂₀-alkyl groups,
straight-chain or branched C₃- to C₂₀-alkenyl groups,
straight-chain or branched C₁- to C₂₀-hydroxyalkyl groups, where the hydroxyl group may be bonded to a primary or secondary carbon atom in the chain and furthermore the alkyl chain may also be interrupted by oxygen, and/or
C₃- to C₁₋₁₀-cycloalkyl groups and/or C₃- to C₁₋₂-cycloalkenyl groups, where the rings may each also be bridged by —(CH₂)ₙ— groups, where n=1 to 3,
where all OR¹¹ are, independently of one another,
   OH
   straight-chain or branched C₁- to C₂₀-alkoxy groups,
   straight-chain or branched C₃- to C₂₀-alkenyloxy groups,
   straight-chain or branched C₁- to C₂₀-hydroxyalkoxy groups, where the hydroxyl group(s) may be bonded to a primary or secondary carbon atom in the chain and furthermore the alkyl chain may also be interrupted by oxygen, and/or
   C₃- to C₁₋₁₀-cycloalkoxy groups and/or C₃- to C₁₋₂-cycloalkenyloxy groups, where the rings may each also be bridged by —(CH₂)ₙ— groups, where n=1 to 3, and/or
   mono- and/or oligoglycosyl radicals,
with the proviso that at least 4 radicals from R¹ to R⁷ are OH and that at least 2 pairs of adjacent —OH groups are present in the molecule,
or R², R⁵ and R⁶ are OH and the radicals R¹, R³, R⁴ and R⁷⁻¹⁰ are H,
   as described in German patent application DE-A-10244282.

Of the coumaranones, preference is given to 4,6,3′,4′-tetrahydroxybenzyl-coumaranone-3.

The term chromone derivatives is preferably taken to mean certain chromen-2-one derivatives which are suitable as active ingredients for the preventative treatment of human skin and human hair against ageing processes and harmful environmental influences. At the same time, they exhibit a low irritation potential for the skin, have a positive effect on water binding in the skin, maintain or increase the elasticity of the skin and thus promote smoothing of the skin. These compounds preferably conform to the formula III

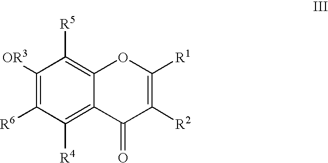

where
R¹ and R² may be identical or different and are selected from
H, —C(═O)—R⁷, —C(═O)—OR⁷,
straight-chain or branched C₁- to C₂₀-alkyl groups,
straight-chain or branched C₃- to C₂₀-alkenyl groups,
straight-chain or branched C₁- to C₂₀-hydroxyalkyl groups, where the hydroxyl group may be bonded to a primary or secondary carbon atom in the chain and furthermore the alkyl chain may also be interrupted by oxygen, and/or
C₃- to C₁₋₁₀-cycloalkyl groups and/or C₃- to C₁₋₂-cycloalkenyl groups, where the rings may each also be bridged by —(CH₂)ₙ— groups, where n=1 to 3,
   R³ is H or straight-chain or branched C₁- to C₂₀-alkyl groups,

### R⁴ is H or OR⁸,

R⁵ and R⁶ may be identical or different and are selected from
—H, —OH,
straight-chain or branched to C₁- to C₂₀-alkyl groups,
straight-chain or branched C₃- to C₂₀-alkenyl groups and
straight-chain or branched C₁- to C₂₀-hydroxyalkyl groups, where the hydroxyl group may be bonded to a primary or secondary carbon atom in the chain and furthermore the alkyl chain may also be interrupted by oxygen, and
   R⁷ is H, straight-chain or branched C₁- to C₂₀-alkyl groups, a polyhydroxyl compound, such as preferably an ascorbic acid radical or glycosidic radicals, and
   R⁸ is H or straight-chain or branched C₁- to C₂₀-alkyl groups, where at least two of the substituents R¹, R² and R⁴—R⁶ are not H or at least one substituent from R¹ and R² is —C(═O)—R⁷ or —C(═O)—OR⁷.

The proportion of one or more compounds selected from flavonoids, chromone derivatives and coumaranones in the composition according to the invention is preferably from 0.001 to 5% by weight, particularly preferably from 0.01 to 2% by weight, based on the composition as a whole.

Particularly preferred active ingredients are, for example, also so-called compatible solutes. These are substances which are involved in the osmo-regulation of plants or microorganisms and can be isolated from these organisms. The generic term compatible solutes here also encompasses the osmolytes described in German patent application DE-A-10133202. Suitable osmolytes are, for example, the polyols, methylamine compounds and amino acids and respective precursors thereof. Osmolytes in the sense of German patent application. DE-A-10133202 are, in particular, substances from the group consisting of the polyols, such as, for example, myo-inositol, mannitol or sorbitol, and/or one or more of the osmolytically active substances mentioned below:

taurine, choline, betaine, phosphorylcholine, glycerophosphorylcholines, glutamine, glycine, α-alanine, glutamate, aspartate and proline. Precursors of these substances are, for example, glucose, glucose polymers, phosphatidylcholine, phosphatidylinositol, inorganic phosphates, proteins, peptides and polyamino acids. Precursors are, for example, compounds which are converted into osmolytes by metabolic steps.

Compatible solutes which are preferably employed in accordance with the invention are substances selected from the group consisting of pyrimidinecarboxylic acids (such as ectoine and hydroxyectoine), proline, betaine, glutamine, cyclic diphosphoglycerate, N-acetylornithine, trimethylamine N-oxide, di-myo-inositol phosphate (DIP), cyclic 2,3-diphosphoglycerate (cDPG), 1,1-diglycerol phosphate (DGP), β-mannosyl glycerate (firoin), β-mannosyl glyceramide (firoin-A) and/or dimannosyl diinositol phosphate (DMIP) or an optical isomer, derivative, for example an acid, a salt or ester, of these compounds, or combinations thereof.

Of the pyrimidinecarboxylic acids, particular mention should be made here of ectoine ((S)-1,4,5,6-tetrahydro-2-methyl-4-pyrimidinecarboxylic acid) and hydroxyectoine ((S,S)-1,4,5,6-tetrahydro-5-hydroxy-2-methyl-4-pyrimidinecarboxylic acid) and derivatives thereof. These compounds stabilise enzymes and other biomolecules in aqueous solutions and organic solvents. Furthermore, they stabilise, in particular, enzymes against denaturing conditions, such as salts, extreme pH values, surfactants, urea, guanidinium chloride and other compounds.

Ectoine and ectoine derivatives, such as hydroxyectoine, can advantageously be used in medicaments. In particular, hydroxyectoine can be employed for the preparation of a medicament for the treatment of skin diseases. Other areas of application of hydroxyectoine and other ectoine derivatives are typically in areas in which, for example, trehalose is used as additive. Thus, ectoine derivatives, such as hydroxyectoine, can be used as protectant in dried yeast and bacterial cells. Pharmaceutical products, such as non-glycosylated, pharmaceutically active peptides and proteins, for example t-PA, can also be protected with ectoine or its derivatives.

Of the cosmetic applications, particular mention should be made of the use of ectoine and ectoine derivatives for the care of aged, dry or irritated skin. Thus, European patent application EP-A-0 671 161 describes, in particular, that ectoine and hydroxyectoine are employed in cosmetic compositions, such as powders, soaps, surfactant-containing cleansing products, lipsticks, rouge, make-up, care creams and sunscreen preparations.

Preference is given here to the use of a pyrimidinecarboxylic acid of the following formula IV

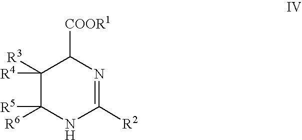

in which R¹ is a radical H or C₁₋₈-alkyl, R² is a radical H or C1-4-alkyl, and R³, R⁴, R⁵ and R⁶ are each, independently of one another, a radical from the group consisting of H, OH, NH₂ and C₁₋₄-alkyl. Preference is given to the use of pyrimidinecarboxylic acids in which R² is a methyl or ethyl group, and R¹ or R⁵ and R⁶ are H. Particular preference is given to the use of the pyrimidinecarboxylic acids ectoine ((S)-1,4,5,6-tetrahydro-2-methyl-4-pyrimidinecarboxylic acid) and hydroxyectoine ((S,S)-1,4,5,6-tetrahydro-5-hydroxy-2-methyl-4-pyrimidinecarboxylic acid). In this case, the compositions according to the invention preferably comprise pyrimidinecarboxylic acids of this type in amounts of up to 15% by weight.

It is particularly preferred in accordance with the invention for the compatible solutes to be selected from di-myo-inositol phosphate (DIP), cyclic 2,3-diphosphoglycerate (cDPG), 1,1-diglycerol phosphate (DGP), β-mannosyl glycerate (firoin), β-mannosyl glyceramide (firoin-A) and/or dimannosyl diinositol phosphate (DMIP), ectoine, hydroxyectoine or mixtures thereof.

Of the aryl oximes likewise preferably employed, preference is given to the use of 2-hydroxy-5-methyllaurophenone oxime, which is also known as HMLO, LPO or F5. Its suitability for use in cosmetic compositions is disclosed, for example, in DE-A-41 16 123. Compositions which comprise 2-hydroxy-5-methyllaurophenone oxime are accordingly suitable for the treatment of skin diseases which are accompanied by inflammation. It is known that compositions of this type can be used, for example, for the therapy of psoriasis, various forms of eczema, irritative and toxic dermatitis, UV dermatitis and further allergic and/or inflammatory diseases of the skin and integumentary appendages. Compositions according to the invention which comprise an aryl oxime, preferably 2-hydroxy-5-methyllaurophenone oxime, exhibit surprising antiinflammatory suitability. The compositions here preferably comprise from 0.01 to 10% by weight of the aryl oxime, it being particularly preferred for the composition to comprise from 0.05 to 5% by weight of aryl oxime.

In a further, likewise preferred embodiment of the present invention, the composition according to the invention comprises at least one self-tanning agent.

Advantageous self-tanning agents which can be employed are, inter alia:

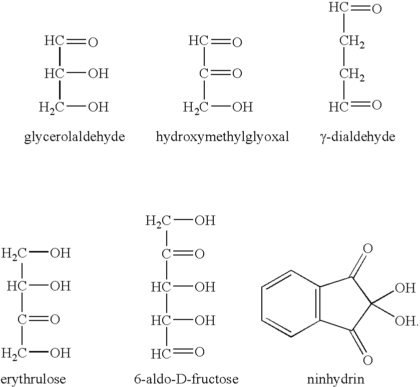

Mention should also be made of 5-hydroxy-1,4-naphthoquinone (juglone), which can be extracted from the shells of fresh walnuts, and 2-hydroxy-1,4-naphthoquinone (lawsone), which occurs in henna leaves. The flavonoid diosmetin and glycosides or sulfates thereof can also be employed. These compounds can be employed in the form of pure substances or plant extracts. Diosmetin can preferably be employed, for example, in the form of a chrysanthemum extract.

Very particular preference is given to 1,3-dihydroxyacetone (DHA), a tri-functional sugar which occurs in the human body, and derivatives thereof

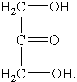

1,3-dihydroxyacetone (DHA).

The said self-tanning agents can be employed alone or in the form of a mixture. It is particularly preferred here for DHA to be employed in a mixture with a further self-tanning agent of those mentioned above.

Furthermore, the compositions according to the invention may also comprise dyes and coloured pigments. The dyes and coloured pigments can be selected from the corresponding positive list in the German Cosmetics Regulation or the EU list of cosmetic colorants. In most cases, they are identical with the dyes approved for foods. Advantageous coloured pigments are, for example, titanium dioxide, mica, iron oxides (for example Fe₂O₃, Fe₃O₄, FeO(OH)) and/or tin oxide. Advantageous dyes are, for example, carmine, Berlin Blue, Chromium Oxide Green, Ultramarine Blue and/or Manganese Violet. It is particularly advantageous to select the dyes and/or coloured pigments from the following list. The Colour Index numbers (CINs) are taken from the Rowe Colour Index, 3rd Edition, Society of Dyers and Colourists, Bradford, England, 1971.

| Chemical or other name | CIN | Colour |
|---|---|---|
| Pigment Green | 10006 | Green |
| Acid Green 1 | 10020 | Green |
| 2,4-dinitrohydroxynaphthalene-7-sulfonic acid | 10316 | Yellow |
| Pigment Yellow 1 | 11680 | Yellow |
| Pigment Yellow 3 | 11710 | Yellow |
| Pigment Orange 1 | 11725 | Orange |
| 2,4-dihydroxyazobenzene | 11920 | Orange |
| Solvent Red 3 | 12010 | Red |
| 1-(2′-chloro-4′-nitro-1′-phenylazo)-2-hydroxynaphthalene | 12085 | Red |
| Pigment Red 3 | 12120 | Red |
| Ceres Red; Sudan Red; Fat Red G | 12150 | Red |
| Pigment Red 112 | 12370 | Red |
| Pigment Red 7 | 12420 | Red |
| Pigment Brown 1 | 12480 | Brown |
| N-(5-chloro-2,4-dimethoxyphenyl)-4-[[5-[(diethylamino)- | 12490 | Red |
| sulfonyl]-2-methoxyphenyl]azo]-3-hydroxynaphthalene-2- | | |
| carboxamide | | |
| Disperse Yellow 16 | 12700 | Yellow |
| 1-(4-Sulfo-1-phenylazo)-4-aminobenzene-5-sulfonic acid | 13015 | Yellow |
| 2,4-dihydroxyazobenzene-4′-sulfonic acid | 14270 | Orange |
| 2-(2,4-dimethylphenylazo-5-sulfonyl)-1-hydroxynaphthalene- | 14700 | Red |
| 4-sulfonic acid | | |
| 2-(4-Sulfo-1-naphthylazo)-1-naphthol-4-sulfonic acid | 14720 | Red |
| 2-(6-Sulfo-2,4-xylylazo)-1-naphthol-5-sulfonic acid | 14815 | Red |
| 1-(4′-Sulfophenylazo)-2-hydroxynaphthalene | 15510 | Orange |
| 1-(2-Sulfonyl-4-chloro-5-carboxy-1-phenylazo)-2- | 15525 | Red |
| hydroxynaphthalene | | |
| 1-(3-Methylphenylazo-4-sulfonyl)-2-hydroxynaphthalene | 15580 | Red |
| 1-(4′,(8′)-Sulfonylnaphthylazo)-2-hydroxynaphthalene | 15620 | Red |
| 2-Hydroxy-1,2′-azonaphthalene-1′-sulfonic acid | 15630 | Red |
| 3-Hydroxy-4-phenylazo-2-naphthylcarboxylic acid | 15800 | Red |
| 1-(2-Sulfo-4-methyl-1-phenylazo)-2-naphthylcarboxylic | 15850 | Red |
| acid | | |
| 1-(2-Sulfo-4-methyl-5-chloro-1-phenylazo)-2-hydroxy- | 15865 | Red |
| naphthalene-3-carboxylic acid | | |
| 1-(2-Sulfo-1-naphthylazo)-2-hydroxynaphthalene-3- | 15880 | Red |
| carboxylic acid | | |
| 1-(3-Sulfo-1-phenylazo)-2-naphthol-6-sulfonic acid | 15980 | Orange |
| 1-(4-Sulfo-1-phenylazo)-2-naphthol-6-sulfonic acid | 15985 | Yellow |
| Allura Red | 16035 | Red |
| 1-(4-Sulfo-1-naphthylazo)-2-naphthol-3,6-disulfonic acid | 16185 | Red |
| Acid Orange 10 | 16230 | Orange |
| 1-(4-Sulfo-1-naphthylazo)-2-naphthol-6,8-disulfonic acid | 16255 | Red |
| 1-(4-Sulfo-1-naphthylazo)-2-naphthol-3,6,8-trisulfonic | 16290 | Red |
| acid | | |
| 8-Amino-2-phenylazo-1-naphthol-3,6-disulfonic acid | 17200 | Red |
| Acid Red 1 | 18050 | Red |
| Acid Red 155 | 18130 | Red |
| Acid Yellow 121 | 18690 | Yellow |
| Acid Red 180 | 18736 | Red |
| Acid Yellow 11 | 18820 | Yellow |
| Acid Yellow 17 | 18965 | Yellow |
| 4-(4-Sulfo-1-phenylazo)-1-(4-sulfophenyl)-5-hydroxy- | 19140 | Yellow |
| pyrazolone-3-carboxylic acid | | |
| Pigment Yellow 16 | 20040 | Yellow |
| 2,6-(4′-Sulfo-2″,4″-dimethyl)bisphenylazo)-1,3-dihydroxy- | 20170 | Orange |
| benzene | | |
| Acid Black 1 | 20470 | Black |
| Pigment Yellow 13 | 21100 | Yellow |
| Pigment Yellow 83 | 21108 | Yellow |
| Solvent Yellow | 21230 | Yellow |
| Acid Red 163 | 24790 | Red |
| Acid Red 73 | 27290 | Red |
| 2-[4′-(4″-Sulfo-1″-phenylazo)-7′-sulfo-1′-naphthylazo]-1- | 27755 | Black |
| hydroxy-7-aminonaphthalene-3,6-disulfonic acid | | |
| 4-[4″-Sulfo-1″-phenylazo)-7′-sulfo-1′-naphthylazo]-1- | 28440 | Black |
| hydroxy-8-acetylaminonaphthalene-3,5-disulfonic acid | | |
| Direct Orange 34, 39, 44, 46, 60 | 40215 | Orange |
| Food Yellow | 40800 | Orange |
| trans-β-Apo-8′-carotene aldehyde (C₃₀) | 40820 | Orange |
| trans-Apo-8′-carotinic acid (C₃₀) ethyl ester | 40850 | Orange |
| Canthaxanthine | 40850 | Orange |
| Acid Blue 1 | 42045 | Blue |
| 2,4-Disulfo-5-hydroxy-4′-4″-bis(diethylamino)triphenyl- | 42051 | Blue |
| carbinol | | |
| 4-[(4-N-Ethyl-p-sulfobenzylamino)phenyl-(4-hydroxy-2- | 42053 | Green |
| sulfophenyl)(methylene)-1-(N-ethyl-N-p-sulfobenzyl)-2,5- | | |
| cyclohexadienimine] | | |
| Acid Blue 7 | 42080 | Blue |
| (N-Ethyl-p-sulfobenzylamino)phenyl-(2-sulfophenyl)- | 42090 | Blue |
| methylene-(N-ethyl-N-p-sulfobenzyl)-Δ^{2,5}- | | |
| cyclohexadienimine | | |
| Acid Green 9 | 42100 | Green |
| Diethyldisulfobenzyldi-4-amino-2-chlorodi-2- | 42170 | Green |
| methylfuchsonimmonium | | |
| Basic Violet 14 | 42510 | Violet |
| Basic Violet 2 | 42520 | Violet |
| 2′-Methyl-4′-(N-ethyl-N-m-sulfobenzyl)amino-4″-(N- | 42735 | Blue |
| diethyl)amino-2-methyl-N-ethyl-N-m-sulfobenzylfuchsonimmonium | | |
| 4′-(N-dimethyl)amino-4″-(N-phenyl)aminonaphtho-N- | 44045 | Blue |
| dimethylfuchsonimmonium | | |
| 2-Hydroxy-3,6-disulfo-4,4′-bisdimethylaminonaphtho- | 44090 | Green |
| fuchsonimmonium | | |
| Acid Red 52 | 45100 | Red |
| 3-(2′-Methylphenylamino)-6-(2′-methyl-4′-sulfophenyl- | 45190 | Violet |
| amino)-9-(2″-carboxyphenyl)xanthenium salt | | |
| Acid Red 50 | 45220 | Red |
| Phenyl-2-oxyfluorone-2-carboxylic acid | 45350 | Yellow |
| 4,5-Dibromofluorescein | 45370 | Orange |
| 2,4,5,7-Tetrabromofluorescein | 45380 | Red |
| Solvent Dye | 45396 | Orange |
| Acid Red 98 | 45405 | Red |
| 3′,4′,5′,6′-Tetrachloro-2,4,5,7-tetrabromofluorescein | 45410 | Red |
| 4,5-diiodofluorescein | 45425 | Red |
| 2,4,5,7-Tetraiodofluorescein | 45430 | Red |
| Quinophthalone | 47000 | Yellow |
| Quinophthalonedisulfonic acid | 47005 | Yellow |
| Acid Violet 50 | 50325 | Violet |
| Acid Black 2 | 50420 | Black |
| Pigment Violet 23 | 51319 | Violet |
| 1,2-dioxyanthraquinone, calcium-aluminium complex | 58000 | Red |
| 3-Oxypyrene-5,8,10-sulfonic acid | 59040 | Green |
| 1-Hydroxy-4-N-phenylaminoanthraquinone | 60724 | Violet |
| 1-Hydroxy-4-(4′-methylphenylamino)anthraquinone | 60725 | Violet |
| Acid Violet 23 | 60730 | Violet |
| 1,4-di(4′-methylphenylamino)anthraquinone | 61565 | Green |
| 1,4-Bis(o-sulfo-p-toluidino)anthraquinone | 61570 | Green |
| Acid Blue 80 | 61585 | Blue |
| Acid Blue 62 | 62045 | Blue |
| N,N′-dihydro-1,2,1′,2′-anthraquinonazine | 69800 | Blue |
| Vat Blue 6; Pigment Blue 64 | 69825 | Blue |
| Vat Orange 7 | 71105 | Orange |
| Indigo | 73000 | Blue |
| Indigodisulfonic acid | 73015 | Blue |
| 4,4′-dimethyl-6,6′-dichlorothioindigo | 73360 | Red |
| 5,5′-dichloro-7,7′-dimethylthioindigo | 73385 | Violet |
| Quinacridone Violet 19 | 73900 | Violet |
| Pigment Red 122 | 73915 | Red |
| Pigment Blue 16 | 74100 | Blue |
| Phthalocyanine | 74160 | Blue |
| Direct Blue 86 | 74180 | Blue |
| chlorinated phthalocyanine | 74260 | Green |
| Natural Yellow 6, 19; Natural Red 1 | 75100 | Yellow |
| Bixin, Nor-Bixin | 75120 | Orange |
| Lycopene | 75125 | Yellow |
| trans-alpha-, -beta- or -gamma-Carotene | 75130 | Orange |
| Keto and/or hydroxyl derivatives of carotene | 75135 | Yellow |
| Guanine or pearlescent agent | 75170 | White |
| 1,7-Bis(4-hydroxy-3-methoxyphenyl)-1,6-heptadiene-3,5- | 75300 | Yellow |
| dione | | |
| Complex salt (Na, Al, Ca) of carminic acid | 75470 | Red |
| chlorophyll a and b; copper compounds of chlorophylls | 75810 | Green |
| and chlorophyllines | | |
| Aluminium | 77000 | White |
| Aluminium hydroxide | 77002 | White |
| Water-containing aluminium silicates | 77004 | White |
| Ultramarine | 77007 | Blue |
| Pigment Red 101 and 102 | 77015 | Red |
| Barium sulfate | 77120 | White |
| Bismuth oxychloride and mixtures thereof with mica | 77163 | White |
| Calcium carbonate | 77220 | White |
| Calcium sulfate | 77231 | White |
| Carbon | 77266 | Black |
| Pigment Black 9 | 77267 | Black |
| Carbo medicinalis vegetabilis | 77268:1 | Black |
| Chromium oxide | 77288 | Green |
| Chromium oxide, water-containing | 77278 | Green |
| Pigment Blue 28, Pigment Green 14 | 77346 | Green |
| Pigment Metal 2 | 77400 | Brown |
| Gold | 77480 | Brown |
| Iron oxides and hydroxides | 77489 | Orange |
| Iron oxide | 77491 | Red |
| Iron oxide hydrate | 77492 | Yellow |
| Iron oxide | 77499 | Black |
| Mixtures of iron(II) and iron(III) hexacyanoferrate | 77510 | Blue |
| Pigment White 18 | 77713 | White |
| Manganese ammonium diphosphate | 77742 | Violet |
| Manganese phosphate; Mn₃(PO₄)₂•7 H₂O | 77745 | Red |
| Silver | 77820 | White |
| Titanium dioxide and mixtures thereof with mica | 77891 | White |
| Zinc oxide | 77947 | White |
| 6,7-dimethyl-9-(1′-D-ribityl)isoalloxazine, lactoflavin | | Yellow |
| Sugar dye | | Brown |
| Capsanthin, capsorubin | | Orange |
| Betanin | | Red |
| Benzopyrylium salts, anthocyans | | Red |
| Aluminium, zinc, magnesium and calcium stearate | | White |
| Bromothymol Blue | | Blue |

It may furthermore be favourable to select, as dye, one or more substances from the following group:

2,4-dihydroxyazobenzene, 1-(2′-chloro-4′-nitro-1′-phenylazo)-2-hydroxynaphthalene, Ceres Red, 2-(4-sulfo-1-naphthylazo)-1-naphthol-4-sulfonic acid, the calcium salt of 2-hydroxy-1,2′-azonaphthalene-1′-sulfonic acid, the calcium and barium salts of 1-(2-sulfo-4-methyl-1-phenylazo)-2-naphthylcarboxylic acid, the calcium salt of 1-(2-sulfo-1-naphthylazo)-2-hydroxynaphthalene-3-carboxylic acid, the aluminium salt of 1-(4-sulfo-1-phenylazo)-2-naphthol-6-sulfonic acid, the aluminium salt of 1-(4-sulfo-1-naphthylazo)-2-naphthol-3,6-disulfonic acid, 1-(4-sulfo-1-naphthylazo)-2-naphthol-6,8-disulfonic acid, the aluminium salt of 4-(4-sulfo-1-phenylazo)-2-(4-sulfophenyl)-5-hydroxypyrazolone-3-carboxylic acid, the aluminium and zirconium salts of 4,5-dibromofluorescein, the aluminium and zirconium salts of 2,4,5,7-tetrabromofluorescein, 3′,4′,5′,6′-tetrachloro-2,4,5,7-tetrabromofluorescein and its aluminium salt, the aluminium salt of 2,4,5,7-tetraiodofluorescein, the aluminium salt of quinophthalonedisulfonic acid, the aluminium salt of indigodisulfonic acid, red and black iron oxide (CIN: 77 491 (red) and 77 499 (black)), iron oxide hydrate (CIN: 77492), manganese ammonium diphosphate and titanium dioxide.

Also advantageous are oil-soluble natural dyes, such as, for example, paprika extract, β-carotene or cochineal.

Also advantageous for the purposes of the present invention are gel creams comprising pearlescent pigments. Particular preference is given to the types of pearlescent pigment listed below:

1. Natural pearlescent pigments, such as, for example,
a) “pearl essence” (guanine/hypoxanthine mixed crystals from fish scales) and
b) “mother-of-pearl” (ground mussel shells)
   2. Monocrystalline pearlescent pigments, such as, for example, bismuth oxychloride (BiOCl)
   3. Layered substrate pigments: for example mica/metal oxide

The basis for pearlescent pigments is formed by, for example, pulverulent pigments or castor oil dispersions of bismuth oxychloride and/or titanium dioxide as well as bismuth oxychloride and/or titanium dioxide on mica. The lustre pigment listed under CIN 77163, for example, is particularly advantageous.

Also advantageous are, for example, the following pearlescent pigment types based on mica/metal oxide:

| | Coating/layer | |
|---|---|---|
| Group | thickness | Colour |
| Silver-white pearlescent pigments | TiO₂: 40-60 nm | Silver |
| Interference pigments | TiO₂: 60-80 nm | Yellow |
| | TiO₂: 80-100 nm | Red |
| | TiO₂: 100-140 nm | Blue |
| | TiO₂: 120-160 nm | Green |
| Coloured lustre pigments | Fe₂O₃ | Bronze |
| | Fe₂O₃ | Copper |
| | Fe₂O₃ | Red |
| | Fe₂O₃ | Red-violet |
| | Fe₂O₃ | Red-green |
| | Fe₂O₃ | Black |
| Combination pigments | TiO₂/Fe₂O₃ | Gold shades |
| | TiO₂/Cr₂O₃ | Green |
| | TiO₂/Berlin Blue | Dark blue |

Particular preference is given to, for example, the pearlescent pigments available from Merck KGaA, Darmstadt, under the trade names Timiron®, Colorona® or Dichrona®.

The list of the said pearlescent pigments is of course not intended to be limiting. Pearlescent pigments which are advantageous for the purposes of the present invention can be obtained by numerous routes known per se. For example, other substrates apart from mica can also be coated with further metal oxides, such as, for example, silica and the like. For example, TiO₂— and Fe₂O₃-coated SiO₂ particles (“Ronasphere” grades), which are marketed by Merck KGaA, Darmstadt, and are particularly suitable for the optical reduction of fine wrinkles, are advantageous.

It may additionally be advantageous to completely omit a substrate such as mica. Particular preference is given to pearlescent pigments prepared using SiO₂. Such pigments, which may additionally also have goniochromatic effects, are available, for example, from BASF AG, Ludwigshafen, under the trade name Sicopearl® Fantastico.

It may also be advantageous to employ Engelhard/Mearl pigments based on calcium sodium borosilicate coated with titanium dioxide. These are available under the name Reflecks®. Due to their particle size of 40-80 μm, they have a glitter effect in addition to the colour.

Also particularly advantageous are effect pigments available from Flora Tech under the trade name Metasomes® Standard/Glitter in various colours (yellow, red, green and blue). The glitter particles here are in the form of mixtures with various assistants and dyes (such as, for example, the dyes with the colour index (CI) numbers 19140, 77007, 77289 and 77491).

The dyes and pigments can be in individual form or in the form of a mixture and mutually coated with one another, with different colour effects generally being caused by different coating thicknesses. The total amount of dyes and colouring pigments is advantageously selected from the range from, for example, 0.1% by weight to 30% by weight, preferably from 0.5 to 15% by weight, in particular from 1.0 to 10% by weight, in each case based on the total weight of the compositions.

All compounds or components which can be used in the compositions are either known and commercially available or can be synthesised by known processes. Any desired conventional carriers, assistants and optionally further active ingredients may be added to the composition.

Preferred assistants originate from the group consisting of preservatives, antioxidants, stabilisers, solubilisers, vitamins, colorants and odour improvers.

Solutions and emulsions may comprise the customary carriers, such as solvents, solubilisers and emulsifiers, for example water, ethanol, isopropanol, ethyl carbonate, ethyl acetate, benzyl alcohol, benzyl benzoate, propylene glycol, 1,3-butyl glycol, oils, in particular cottonseed oil, peanut oil, wheatgerm oil, olive oil, castor oil and sesame oil, glycerol fatty acid esters, polyethylene glycols and fatty acid esters of sorbitan, or mixtures of these substances.

In a preferred embodiment, the compositions according to the invention comprise hydrophilic surfactants.

The hydrophilic surfactants are preferably selected from the group consisting of the alkylglucosides, acyl lactylates, betaines and coconut amphoacetates. The alkylglucosides are themselves advantageously selected from the group consisting of the alkylglucosides which are distinguished by the structural formula

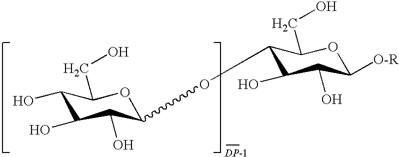

where R is a branched or unbranched alkyl radical having from 4 to 24 carbon atoms, and where D̅P̅ denotes a mean degree of glucosylation of up to 2.

The value D̅P̅ represents the degree of glucosidation of the alkylglucosides used in accordance with the invention and is defined as

$$\overline{DP} = \frac{{p}_{1}}{100} \cdot 1 + \frac{{p}_{2}}{100} \cdot 2 + \frac{{p}_{3}}{100} \cdot 3 + … = \sum \frac{{p}_{i}}{100} \cdot i$$

in which p₁, p₂, p₃ . . . pᵢ, represent the proportion of mono-, di-, tri- . . . i-fold glucosylated products in percent by weight. Products which are advantageously selected in accordance with the invention are those having degrees of glucosylation of 1-2, particularly advantageously of from 1.1 to 1.5, very particularly advantageously of 1.2-1.4, in particular of 1.3.

The value DP takes into account the fact that alkylglucosides are, as a consequence of their preparation, generally mixtures of mono- and oligoglucosides. A relatively high content of monoglucosides, typically in the order of 40-70% by weight, is advantageous in accordance with the invention.

Alkylglycosides which are particularly advantageously used are selected from the group consisting of octyl glucopyranoside, nonyl glucopyranoside, decyl glucopyranoside, undecyl glucopyranoside, dodecyl glucopyranoside, tetradecyl glucopyranoside and hexadecyl glucopyranoside.

It is likewise advantageous to employ natural or synthetic raw materials and assistants or mixtures which are distinguished by an effective content of the active ingredients used, for example Plantaren® 1200 (Henkel KGaA), Oramix® NS 10 (Seppic).

The acyllactylates are themselves advantageously selected from the group consisting of the substances which are distinguished by the structural formula

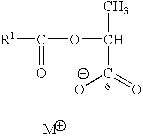

where R¹ denotes a branched or unbranched alkyl radical having from 1 to 30 carbon atoms, and M⁺ is selected from the group consisting of alkali metal ions and the group consisting of ammonium ions which are substituted by one or more alkyl and/or one or more hydroxyalkyl radicals, or corresponds to half an equivalent of an alkaline earth metal ion.

For example, sodium isostearyl lactylate, for example the product Pathionic® ISL from the American Ingredients Company, is advantageous.

The betaines are advantageously selected from the group consisting of the substances which are distinguished by the structural formula

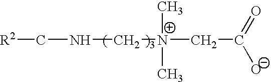

where R² denotes a branched or unbranched alkyl radical having from 1 to 30 carbon atoms.

R² particularly advantageously denotes a branched or unbranched alkyl radical having from 6 to 12 carbon atoms.

For example, capramidopropylbetaine, for example the product Tego® Betain 810 from Th. Goldschmidt AG, is advantageous.

A coconut amphoacetate which is advantageous for the purposes of the invention is, for example, sodium coconut amphoacetate, as available under the name Miranol® Ultra C32 from Miranol Chemical Corp.

The compositions according to the invention are advantageously characterised in that the hydrophilic surfactant(s) is (are) present in concentrations of 0.01-20% by weight, preferably 0.05-10% by weight, particularly preferably 0.1-5% by weight, in each case based on the total weight of the composition.

For use, the cosmetic and dermatological compositions according to the invention are applied in sufficient amount to the skin and/or hair in the usual manner for cosmetics.

The composition may comprise cosmetic adjuvants which are usually used in this type of composition, such as, for example, thickeners, softeners, moisturisers, surfactants, emulsifiers, preservatives, antifoams, perfumes, waxes, lanolin, propellants, dyes and/or pigments which colour the composition itself or the skin, and other ingredients usually used in cosmetics.

The dispersant or solubiliser used can be an oil, wax or other fatty substance, a lower monoalcohol or lower polyol or mixtures thereof. Particularly preferred monoalcohols or polyols include ethanol, isopropanol, propylene glycol, glycerol and sorbitol.

A preferred embodiment of the invention is an emulsion in the form of a protective cream or milk which comprises, for example, fatty alcohols, fatty acids, fatty acid esters, in particular triglycerides of fatty acids, lanolin, natural and synthetic oils or waxes and emulsifiers in the presence of water. Hydrogels are also a preferred embodiment.

If a composition is formulated as an aerosol, the customary propellants, such as alkanes, fluoroalkanes and chlorofluoroalkanes, are generally used.

The cosmetic composition may also be used to protect the hair against photochemical damage in order to prevent changes of colour shade, bleaching or damage of a mechanical nature. In this case, a suitable formulation is in the form of a rinse-out shampoo, lotion, gel or emulsion, the composition in question being applied before or after shampooing, before or after colouring or bleaching or before or after permanent waving. It is also possible to select a composition in the form of a lotion or gel for styling or treating the hair, in the form of a lotion or gel for brushing or blow-waving the hair, in the form of a hair lacquer, permanent waving composition, colorant or bleach for the hair. The composition having light-protection properties may comprise various adjuvants used in this type of composition, such as surfactants, thickeners, polymers, softeners, preservatives, foam stabilisers, electrolytes, organic solvents, silicone derivatives, oils, waxes, anti-grease agents, dyes and/or pigments which colour the composition itself or the hair, or other ingredients usually used for hair care.

The compositions according to the invention can be prepared here with the aid of techniques which are well known to the person skilled in the art. The substances according to the invention can be incorporated directly into cosmetic compositions without further preparatory measures.

The compositions according to the invention may advantageously, as already described above, comprise further UV filter substances, where the total amount of the filter substances is, for example, from 0.1% by weight to 30% by weight, preferably from 0.5 to 10% by weight, in particular from 1 to 6% by weight, based on the total weight of the compositions.

Furthermore, the compositions according to the invention can also be used as pharmaceutical compositions for the preventative treatment of inflammation and allergies of the skin and also in certain cases for the prevention of certain types of cancer. The pharmaceutical compositions according to the invention can be administered orally or topically.

The compositions according to the invention can be prepared with the aid of techniques which are well known to the person skilled in the art. Even without further comments, it is assumed that a person skilled in the art will be able to utilise the above description in its broadest scope. The preferred embodiments should therefore merely be regarded as descriptive disclosure which is absolutely not limiting in any way. The following examples are intended to illustrate the present invention without restricting it. All amount data, proportions and percentages are, unless stated otherwise, based on the weight and the total amount or total weight of the compositions. The complete disclosure content of all applications and publications mentioned above and below is incorporated into this application by way of reference. In these examples, the preferred compound of the formula I used is the N,N-dimethyldecanamide Spectrasolv DMDA (Jiangsu Feixang Chemicals or Lehmann & Voss). The preferred sparingly soluble organic UV filter is Ethylhexyl Triazone.

### EXAMPLES

### In General: Determination of the Capsule Size

The particle size determination is carried out by means of laser diffraction in accordance with ISO/DIS 13320, under the conditions as follows:

Instrument: Malvern Mastersizer 2000 with Hydro 2000G dispersion unit The sample weighing is carried out as direct addition using a disposable pipette.
Dispersion medium: water
Sample preparation: none
Dispersion aid: none
Stirrer speed: 800 rpm
Pump speed: 1800 rpm
Ultrasound: none
Refractive index of particles: 1.55

### Absorption: 0.001

Refractive index of dispersion medium: 1.33
Measurement time [sec/snaps]: 5/5000
Background measurement time [sec/snaps]: 8/8000
Number of measurements: 1

### Obscuration [%]: 8-12

The in vivo SPF (sun protection factor) was measured by the international method as published in COLIPA 001-2003—see in detail Example 6. [COLIPA the European cosmetic toiletry and perfumery association]

The in vitro (UV)APF was determined in accordance with German standard DIN 67502, “Characterisation of the UVA protective action of dermal sunscreens by transmission measurements taking into account the light protection factor”, 3rd draft standard, March 2003.

### Example 1

A solution of 400 g of Ethylhexyl Triazone, 400 g of Dimethyl Capramide (Spectrasolv DMDA) and 240 g of tetraethyl orthosilicate is emulsified in a surfactant solution [448 g of deionised water and 11 g of cetyltrimethylammonium chloride (CTAC)] with cooling with the aid of an emulsification tool (Ultra Turax). The finished emulsion is added to water containing hydrochloric acid with stirring. The mixture obtained is stirred at room temperature for 48-72 h. The ethanol formed on hydrolysis of the alkylsilane is then reduced in concentration by distillation. After addition of 20 g of PVP in 300 g of deionised water, the pH of the residue is adjusted to 3.4-3.6 using Na citrate solution, and the mixture is made up with deionised water.

The active-ingredient content of the suspension is 18% by weight.

Incorporation into the cosmetic composition can take place in this form.

The silica capsule can be isolated by conventional methods.

Particle size: d(0.50)=0.53 μm, d(0.90)=1.39 μm

### Example 2

In comparison to Example 1, UV filter capsules were produced using solvents known on the market.

The solubility of Ethylhexyl Triazone in the systems indicated can be determined as follows:

The test substance is initially incorporated into the solvent (for example cosmetic emollient, as indicated in the table) at room temperature (about 20° C.-25° C.) with stirring in small beakers on a heatable magnetic stirrer (the stirring time here should not exceed about 30 min.). The assessment is carried out visually, i.e. it is checked whether a completely clear solution and without evident particles is present. The amounts, conditions and stirring times employed should be noted. If the substance is readily soluble, the concentration is increased by subsequent incorporation of further substance, for example by addition in 0.1 g steps with stirring. The batch size is usually 10 g, i.e. for a solubility of 1% (w/w): 0.1 g of test substance in 9.9 g of solvent. The assessment is carried out visually, if necessary with the aid of a microscope with lambda plate.

Determination of the content of the UV filters: HPLC, determination using external standard. Chromatographic system: stainless steel cartridge LiChroCART® 250 mm 4 mm, Superspher® 100 RP-18 end-capped, particle size 4 μm. Column temperature: room temperature.

Mobile phase: methanol/solution A (80:20); flow rate 1.0 ml/min. Detection: UV detector.

### Sample Preparation:

Sample solution: About 30 mg of the product, weighed accurately, are dispersed with 40 ml of methanol in a 100 ml volumetric flask. This mixture is mixed in an ultrasound bath for 5 minutes. After cooling, the mixture is made up to the mark with methanol. The mixture is diluted correspondingly for the measurement. Before injection, the mixture is filtered using an Anotop® 25 syringe filter (pore size 0.02 μm).
Comparative solution: About 30 mg of Ethylhexyl Triazone, weighed accurately, are dissolved in methanol in a 100 ml volumetric flask, and the solution is made up to the mark. 1.0 ml of this solution is diluted to 50 ml with methanol in a 50 ml volumetric flask.

The evaluation is carried out, for example, using Agilent HPLC Chem-Station; i.e. the peak areas are evaluated by the method of external standard evaluation.

Solubilities of Ethylhexyl Triazone at room temperature (20° to 25° C.) in various solvents and content in the aqueous dispersion

| | Ethylhexyl Triazone | |
|---|---|---|
| Solvent | solubility at 25° C. | Content* |
| Dioctyl Malate | 13% | 4.4% |
| C12-13 Alkyl Lactate | 22% | 7.5% |
| Di-C12-13 Alkyl Tartrate | 35% | 11.9% |
| Spectrasolv DMDA | about 55% | 18.7% |

*at a capsule content of 34% and 60% of H₂O

The contents that can be achieved with the emollient according to the invention, of a maximum of 18.7% at a capsule content of 34% and a water content of 60%, are significantly higher, about 57% higher, than in the case of the previous prior art.

### Example 3

A solution of 240 g of Bis-Ethylhexyloxyphenol Methoxyphenyl Triazine, 560 g of Dimethyl Capramide (Spectrasolv DMDA) and 240 g of tetraethyl orthosilicate is emulsified in a surfactant solution [448 g of deionised water and 11 g of cetyltrimethylammonium chloride (CTAC)] with the aid of an emulsification tool (Ultra Turrax) with cooling. The finished emulsion is added to water containing hydrochloric acid with stirring. The mixture obtained is stirred at room temperature for 48-72 h. The ethanol formed on hydrolysis of the alkylsilane is then reduced in concentration by distillation. After addition of 20 g of PVP in 300 g of deionised water, the pH of the residue is adjusted to 3.4-3.6 using Na citrate solution, and the mixture is made up with deionised water.

The active-ingredient content of the suspension is 10.8% by weight.

Incorporation into the cosmetic composition can take place in this form.

The silica capsule can be isolated by conventional methods.

Particle size: d(0.50)=0.53 μm, d(0.90)=1.39 μm

### Example 4

### Formulation Examples

### A) Hydrogel

| | | 43-06-H-2 | |
|---|---|---|---|
| Raw material | INCI | [%] | [g] |
| UV filter capsule | | 10.00 | 20.00 |
| (17.5% of Ethylhexyl | | | |
| Triazone in | | | |
| Spectrasolv DMDA) | | | |
| Lubrajel DV | PROPYLENE GLYCOL, | 20.00 | 40.00 |
| | POLYGLYCERYL- | | |
| | METHACRYLATE | | |
| RonaCare ® ectoin | ECTOIN | 0.50 | 1.00 |
| Water, | AQUA (WATER) | 68.80 | 137.60 |
| demineralised | | | |
| Germaben II | PROPYLENE GLYCOL, | 0.70 | 1.40 |
| | DIAZOLIDINYL UREA, | | |
| | METHYLPARABEN, | | |
| | PROPYLPARABEN | | |
| | | 100.00 | 200.00 |

### Preparation:

Initially introduce Lubrajel, and add the other constituents with stirring.
In vivo SPF: 4.6; UVA-PF: 1

### B) Oil-in-Water

| 0.1% of Neolone | | | |
|---|---|---|---|
| 950 | | 43.06-OW-2 | |
| Raw material | INCI | [%] | [g] |
| A | | | |
| Eusolex ® 9020 | BUTYL, | 2.00 | 5.00 |
| | METHOXYDIBENZOYL- | | |
| | METHANE | | |
| Paraffin, viscous | PARAFFINUM | 4.50 | 11.25 |
| | LIQUIDUM | | |
| | (MINERAL OIL) | | |
| Pelemol BIP | ISOPROPYLPHTALIMIDE, | 6.00 | 15.00 |
| | BUTYLPHTALIDE | | |
| Isopropyl palmitate | ISOPROPYL PALMITATE | 7.50 | 18.75 |
| Soya oil | GLYCINE SOJA | 5.00 | 12.50 |
| | (SOYBEAN OIL) | | |
| RonaCare ® | TOCOPHERYL ACETATE | 1.00 | 2.50 |
| tocopherol acetate | | | |
| Carbopol Ultrez 10 | CARBOMER | 0.30 | 0.75 |
| B | | | |
| UV filter capsule | | 27.00 | 67.50 |
| (17.5% of | | | |
| Ethylhexyl | | | |
| Triazone in | | | |
| Spectrasolv | | | |
| DMDA) | | | |
| Water, | AQUA (WATER) | 38.50 | 96.25 |
| demineralised | | | |
| Sisterna L70-C | AQUA (WATER), SUCROSE | 6.00 | 15.00 |
| | LAURATE, ALCOHOL | | |
| Phenonip | PHENOXYETHANOL, | 1.00 | 2.50 |
| | BUTYLPARABEN, | | |
| | ETHYLPARABEN, | | |
| | PROPYLPARABEN; | | |
| | METHYLPARABEN | | |
| C | | | |
| Sodium hydroxide | SODIUM HYDROXIDE | 1.20 | 3.00 |
| solution, 10% | | | |
| | | 100.00 | 250.00 |

### Preparation

Combine phase A apart from the Carbopol, and dissolve the Eusolex® 9020. If necessary, warm to about 50° C. Incorporate the Carbopol, and emulsify in the predissolved phase B with stirring. Homogenise. After addition of phase C, homogenise briefly again.
In vivo SPF: 14.3; UVA-PF: 4.5

### C) Water-in-Oil

| | | 43-06-WO-2 | |
|---|---|---|---|
| Ingredient | INCI | [%] | [g] |
| A | | | |
| Eusolex ® T-2000 | TITANIUM DIOXIDE, | 10.00 | 25.00 |
| | ALUMINA, | | |
| | SIMETHICONE | | |
| Arlacel P135 | PEG-30 | 2.50 | 6.25 |
| | DIPOLYHYDROXY- | | |
| | STEARATE | | |
| Abil WE 09 | POLYGLYCERYL-4 | 2.50 | 6.25 |
| | ISOSTEARATE, | | |
| | CETYL PEG/PPG- | | |
| | 10/1 DIME | | |
| Cetiol A | HEXYL LAURATE | 10.00 | 25.00 |
| Cetiol 868 | ETHYLHEXYL STEARATE | 14.00 | 35.00 |
| Shea butter | BUTYROSPERMUM | 1.00 | 2.50 |
| | PARKII | | |
| | (SHEA BUTTER) | | |
| Paracera M | MICROWAX | 0.50 | 1.25 |
| Crodafos CES | CETEARYL, ALCOHOL, | 1.00 | 2.50 |
| | DICETYL | | |
| | PHOSPHATE, | | |
| | CETETH-10-PH | | |
| Dow Corning 200 | DIMETHICONE | 1.00 | 2.50 |
| (100cs) | | | |
| Dow Corning 345 | CYCLOMETHICONE | 1.00 | 2.50 |
| B | | | |
| UV filter capsule | | 18.90 | 47.25 |
| (17.5% of Ethylhexyl | | | |
| Triazone in | | | |
| Spectrasolv DMDA) | | | |
| RonaCare ® ectoin | ECTOIN | 0.30 | 0.75 |
| Rona Care ® allantoin | ALLANTOIN | 0.20 | 0.50 |
| Propylene glycol, 1,2- | SODIUM CHLORIDE | 0.40 | 1.00 |
| Sodium chloride | | | |
| Titriplex ® III | DISODIUM EDTA | 0.05 | 0.13 |
| Water, demineralised | AQUA (WATER) | 32.95 | 82.38 |
| C | | | |
| Phenonip | PHENOXYETHANOL, | 0.70 | 1.75 |
| | BUTYLPARABEN, | | |
| | ETHYLPARABEN, PRO | | |
| | | 100.00 | 250.00 |

### Preparation

Phase A is combined, without Eusolex® T-2000, and heated to 80° C. Eusolex® T-2000 is then slowly added to the hot oil phase with stirring. Phase B is prepared and heated to 75° C. Phase B is then slowly added to phase A with stirring. Homogenise and cool. Phase C is added below 35° C.
In vivo SPF: 35.4; UVA-PF: 4.5

### D-1) Oil-in-Water

| | | Emulsion 1 | Emulsion 2 |
|---|---|---|---|
| | | comprising 2% of | comprising 4% of |
| | | Ethylhexyl Triazone | Ethylhexyl Triazone |
| Trade Name | INCI | [%] | [%] |
| A | | | |
| Cetiol B | Dibutyl Adipate | 9.00 | 8.00 |
| Tegosoft TN | C12-15 Alkyl Benzoate | 9.00 | 8.00 |
| Myritol 331 | Cocoglycerides | 12.00 | 12.00 |
| Eumulgin VL 75 | Lauryl Glucoside, | 4.00 | 4.00 |
| | Polyglyceryl-2- | | |
| | Dipolyhydroxystearate, | | |
| | Glycerin | | |
| Lanette O | Cetearyl Alcohol | 2.00 | 2.00 |
| Uvinul T 150 | Ethylhexyl Triazone | | 2.00 |
| B | | | |
| Glycerin 87% | Glycerin | 3.00 | 3.00 |
| Titriplex III | Disodium EDTA | 0.10 | 0.10 |
| Cremophor | Ceteareth-25 | 1.00 | 1.00 |
| A 25 | | | |
| Keltrol RD | Xanthan Gum | 0.30 | 0.30 |
| Veegum Ultra | Magnesium Aluminium | 1.50 | 1.50 |
| | Silicate | | |
| Water | Aqua (water) | to 100.00 | to 100.00 |
| C | | | |
| Citric acid | Citric Acid | 0.50 | 0.50 |
| UV filter | Aqua (water), | 12.10 | 12.10 |
| capsule | Ethylhexyl Triazone, | | |
| (16.6% of | Dimethyl Capramide, | | |
| Ethylhexyl | Silica, PVP, | | |
| Triazone in | Chlorphenesin | | |
| Spectrasolv | | | |
| DMDA) | | | |
| corresponds to | | | |
| 2% of | | | |
| Uvinul ® T 150 | | | |
| D | | | |
| Phenonip | Phenoxyethanol, | 1.00 | 1.00 |
| | Methylparaben, | | |
| | Ethylparaben, | | |
| | Butylparaben, | | |
| | Propylparaben, | | |
| | Isobutylparaben | | |

### Preparation:

Combine phase A and heat to 80° C. Check whether the solid UV filter has dissolved.
Combine phase B and likewise heat to 80° C. Emulsify phase B into phase A with stirring and homogenise for 3 min. Cool with stirring, adjust the pH using citric acid, and, at about 30° C., incorporate Eusolex® UV-Pearls™, Uvinul T 150 and preservatives.

### D-2) Oil-in-Water

| Raw material | INCI | [%] | [g] |
|---|---|---|---|
| A | | | |
| Tinosorb S | BIS- | 2.00 | 5.00 |
| | ETHYLHEXYLOXYPHENOL | | |
| | METHOXYPHENYL | | |
| | TRIAZINE | | |
| Spectrasolv DMDA | DIMETHYL CAPRAMIDE | 7.00 | 25.00 |
| Paraffin, viscous | PARAFFINUM LIQUIDUM | 3.00 | 5.00 |
| | (MINERAL OIL) | | |
| Pelemol BIP | ISOPROPYLPHTALIMIDE, | 6.00 | 15.00 |
| | BUTYLPHTALIDE | | |
| Soya oil | GLYCINE SOJA (SOYBEAN | 5.00 | 12.50 |
| | OIL) | | |
| RonaCare ® | TOCOPHERYL ACETATE | 1.00 | 2.50 |
| tocopherol acetate | | | |
| Carbopol Ultrez 10 | CARBOMER | 0.30 | 0.75 |
| B | | | |
| UV filter capsule | | 27.00 | 67.50 |
| (10.5% of Tinosorb | | | |
| S in Spectrasolv | | | |
| DMDA) | | | |
| Water, | AQUA (WATER) | to 100 | to 250 |
| demineralised | | | |
| Sisterna L70-C | AQUA (WATER), SUCROSE | 6.00 | 15.00 |
| | LAURATE, ALCOHOL | | |
| Phenonip | PHENOXYETHANOL, | 1.00 | 2.50 |
| | BUTYLPARABEN, | | |
| | ETHYLPARABEN, | | |
| | PROPYLPARABEN; | | |
| | METHYLPARABEN | | |
| C | | | |
| Sodium hydroxide | SODIUM HYDROXIDE | 1.20 | 3.00 |
| solution, 10% | | | |
| | | 100.00 | 250.00 |

### Preparation

Combine phase A apart from the Carbopol and dissolve the Tinosorb S. If necessary, warm to about 60 C. Incorporate the Carbopol and emulsify the predissolved phase B with stirring. Homogenise. After addition of phase C, homogenise briefly again.
In vivo SPF:10; UVA-PF:7.1

### E) Water-in-Oil

| Ingredient | INCI | [%] | [g] |
|---|---|---|---|
| A | | | |
| Tinosorb S | BIS- | 2.00 | 5.00 |
| | ETHYLHEXYLOXYPHENOL | | |
| | METHOXYPHENYL | | |
| | TRIAZINE | | |
| Spectrasolv DMDA | DIMETHYL CAPRAMIDE | 7.00 | 25.00 |
| Eusolex ® T-2000 | TITANIUM DIOXIDE, | 10.00 | 25.00 |
| | ALUMINA, | | |
| | SIMETHICONE | | |
| Arlacel P135 | PEG-30 | 2.50 | 6.25 |
| | DIPOLYHYDRO- | | |
| | XYSTEARATE | | |
| Abil WE 09 | POLYGLYCERYL-4 | 2.50 | 6.25 |
| | ISOSTEARATE, CETYL PEG/ | | |
| | PPG-10/1 DIME | | |
| Cetiol A | HEXYL LAURATE | 3.00 | 7.5 |
| Cetiol 868 | ETHYLHEXYL STEARATE | 14.00 | 35.00 |
| Shea butter | BUTYROSPERMUM PARKII | 1.00 | 2.50 |
| | (SHEA BUTTER) | | |
| Paracera M | MICROWAX | 0.50 | 1.25 |
| Crodafos CES | CETEARYL, ALCOHOL, | 1.00 | 2.50 |
| | DICETYL PHOSPHATE, | | |
| | CETETH-10-PH | | |
| Dow Corning 200 | DIMETHICONE | 1.00 | 2.50 |
| (100cs) | | | |
| Dow Corning 345 | CYCLOMETHICONE | 1.00 | 2.50 |
| B | | | |
| UV filter capsule | | 18.90 | 47.25 |
| (10.5% of Tinosorb | | | |
| S in Spectrasolv | | | |
| DMDA) | | | |
| RonaCare ® ectoin | ECTOIN | 0.30 | 0.75 |
| Rona Care ® | ALLANTOIN | 0.20 | 0.50 |
| allantoin | | | |
| Propylene glycol, | SODIUM CHLORIDE | 0.40 | 1.00 |
| 1,2-Sodium chloride | | | |
| Titriplex ® III | DISODIUM EDTA | 0.05 | 0.13 |
| Water, | AQUA (WATER) | to 100 | to 250 |
| demineralised | | | |
| C | | | |
| Phenonip | PHENOXYETHANOL, | 0.70 | 1.75 |
| | BUTYLPARABEN, | | |
| | ETHYLPARABEN, | | |
| | PROPYLPARABEN | | |
| | | 100.00 | 250.00 |

### Preparation

Phase A is combined with stirring, without Eusolex® T-2000, and heated at 80° C. until the Tinosorb S has dissolved. Eusolex® T-2000 is then slowly added to the hot oil phase with stirring. Phase B is prepared and heated to 75° C. Phase B is then slowly added to phase A with stirring. Homogenise and cool. Phase C is added below 35° C.
In vivo SPF:36.5; UVA-PF:12

### F) Water-in-Oil

| Ingredient | INCI | [%] | [g] |
|---|---|---|---|
| A | | | |
| Tinosorb S | BIS-ETHYLHEXYLOXYPHENOL | 2.00 | 5.00 |
| | METHOXYPHENYL TRIAZINE | | |
| Spectrasolv DMDA | DIMETHYL CAPRAMIDE | 7.00 | 25.00 |
| Eusolex ® T-2000 | TITANIUM DIOXIDE, ALUMINA, | 4.00 | 10.00 |
| | SIMEETHICONE | | |
| RonaCare ® AP | BIS-ETHYLHEXYL | 2.00 | 5.00 |
| | HYDROXYDIMETHOXY | | |
| | BENZYLMALONATE | | |
| Imwitor 372 P | GLYCERYL STEARATE CITRATE | 3.50 | 8.75 |
| Imwitor 380 | GLYCERYL COCOATE CITRATE | 2.00 | 5.00 |
| Wheat germ oil, | TRITICUM VULGARE (WHEAT | 2.00 | 5.00 |
| refined | GERM OIL) | | |
| Propyl | PROPYLPARABEN | 0.05 | 0.13 |
| 4-hydroxybenzoate | | | |
| B | | | |
| UV filter capsule | | | |
| (10.5% of Tinosorb S | | 20.00 | 50.00 |
| in Spectrasolv DMDA) | | | |
| Karion F liquid | SORBITOL | 3.00 | 7.50 |
| Keltrol SF | XANTHAN GUM | 0.50 | 1.25 |
| Methyl | METHYLPARABEN | 0.15 | 0.37 |
| 4-hydroxybenzoate | | | |
| Titriplex ® III | DISODIUM EDTA | 0.05 | 0.13 |
| Water, demineralised | AQUA (WATER) | to 100 | to 250 |
| C | | | |
| Sodium hydroxide | AQUA (WATER), SODIUM | 0.20 | 0.50 |
| solution, 10% | HYDROXIDE | | |
| | | to 100.00 | to 250.00 |

### Preparation

Phase B: Disperse the Keltrol in water. Add the remaining constituents and mix.
Heat phases A and B separately to 80° C.
Emulsify phase B into phase A. Homogenise.
Allow to cool with stirring and adjust the pH to about 6.0 using phase C at below 35° C.
In vivo SPF:21.3; UVA-PF: 9.9

### Example 5

Determination of the in vitro SPF.

Determination of the in vitro SPF of cosmetic compositions:

The basic measurement principle on which the measurement is based is the determination of the UV transmission by a composition which comprises substances for protection against UV light. The composition here is applied to a suitable substrate in a defined layer thickness, and the absorption is measured in nanometre steps in a suitable UV photometer. The in vitro light protection factor is calculated from the following formula:

$$\begin{matrix}{SPF}_{vitro} = \frac{{\int }_{290 n m}^{400 n m} E ( λ ) * S ( λ ) * δ ( λ )}{{\int }_{290 n m}^{400 n m} E ( λ ) * S ( λ ) * δ ( λ ) / MPF ( λ )} \\ = \frac{{\int }_{290 n m}^{400 n m} E ( λ ) * S ( λ ) * δ ( λ )}{{\int }_{290 n m}^{400 n m} E ( λ ) * S ( λ ) * δ ( λ ) / {10}^{A ( λ )}}\end{matrix}$$

E(λ)=irradiation strength at wavelength λ of the reference sunlight spectrum
S(λ)=erythemal effectiveness at wavelength λ
MPF(λ)=monochromatic protection factor
A(λ)=absorption
Substrate: PMMA Plexiglass plates, type XT220070 with a size of 7.5 cm×2.5 cm (Roehm, Darmstadt), roughened on one side by sand blasting (90-150 μm glass beads, 30 bar, 30 cm separation). Specification: DIN 67502
Application rate: 1.25 mg/cm²±5%
Sample preparation: as many small droplets as possible of the sample to be determined are applied to the substrate using a suitable pipette or a spatula and distributed homogeneously. The tare weight of the substrate, the application rate in the moist state and after equilibration (20 min at room temperature) should be noted here. The equilibration is carried out in the dark. Each sample is measured on at least three substrates.

The subsequent measurement of the absorption is carried out by UV photometry (for example Cary 300 Bio (Varian Inc., Palo Alto, USA) with an Ulbricht sphere (Labsphere DRA-CA-301, North Suttin, USA)) over a measurement range of 290-400 nm in 1 nm steps. The spectral band width is 2 nm.

Before measurement of the sample, a base line should be recorded, using a substrate without sample (100% transmission). The subsequent measurement of the transmission (or absorption) of the samples is carried out at four measurement points per plate. The total of 12 measurement points per sample are evaluated by means of suitable software (for example Excel).

In vitro SPF [PMMA, 0.75 mg/cm²]

### Example C=23.0

### Example D=4.5.

### Example 6

### Determination of the SPF Value In Vivo in Accordance with COLIPA (International Sun Protection Factor (SPF) Test Method, COLIPA, May 2006)

UV source: 300 watt xenon arc lamp solar simulator (model 601-300 Multi-port, Solar Light Co. Inc. Philadelphia, Pa., USA)

### Method

Preliminary test for determination of the MED (minimum erythemal dose)
6 different UV irradiation doses (spot 1 cm in diameter) are used on the back area of the test subjects. The MED is in each case determined visually on the unprotected skin of the test subject 16 to 24 hours after irradiation.

### Main Test

The main test is carried out on in each case a 35 cm² area of the back.

Product Application and Standard

70 mg of product are applied to each area in order to give a product amount of 2 mg/cm² (+/−2.5%), with the product being distributed using a glove.

### Waiting Time

After completion of application of the product, a waiting time of 15 minutes is observed before the irradiation is begun.

Before the irradiation, each test area is divided into 6 zones. Each zone is exposed to a different irradiation dose. The irradiation times for the individual test subjects are defined on the basis of the individual MED values determined in the preliminary tests.

The evaluation is carried out by trained personnel 16-24 hours after irradiation.

### Results

For determination of an SPF value, in each case the results determined on 6 test subjects are used. The SPF values are indicated for the following formulations.

### Formulation 1:

| | [% by | | |
|---|---|---|---|
| Phase | wt.] | Raw materials | INCI |
| A | 10.0 | Miglyol 812 | Caprylic/Capric Triglycerides |
| | 1.5 | Abil350 | Dimethicone |
| | 3.0 | Finsolv TN | C12-15 Alkyl Benzoate |
| | 1.0 | Cremophor CO 40 | PEG-40 Hydrogenated Castor |
| | | | Oil |
| B | 12.05 | UV filter capsule | Aqua (water), Ethylhexyl |
| | | (16.6% of Ethylhexyl | Triazone, Dimethyl |
| | | Triazone in Spectrasolv | Capramide, Silica, PVP, |
| | | DMDA) | Chlorphenesin |
| C | 5.0 | Propylene glycol | Propylene Glycol |
| | 5.0 | Ethanol | |
| | 0.5 | Cremophor A 25 | Alcohol Ceteareth-25 |
| | to 100 | Water | Water |
| D | 2.0 | Sepigel 305 | Polyacrylamide, C13-14 |
| | | | Isoparaffin, Laureth-7 |
| E | 1.0 | Euxyl K300 | Phenoxyethanol and |
| | | | Methylparaben and |
| | | | Butylparaben |
| | | | and Ethylparaben and |
| | | | Propylparaben and |
| | | | Isobutylparaben |

### Preparation:

The components of phase A are heated to 80° C.
The components of phase B are dispersed at room temperature with stirring and added to phase A.
The components of phase C are homogenised and added to the mixture of phases A and B and homogenised again.
The components of phases D and E are added to the mixture of phases A+B+C, homogenised again and subsequently cooled to room temperature.
Viscosity: 42600 mPas
pH: 6.5
SPF in vivo according to COLIPA 6.0.

### Formulation 2:

| Phase | [% by wt.] | Raw materials | INCI |
|---|---|---|---|
| A | 10.0 | Miglyol 812 | Caprylic/Capric Triglycerides |
| | 1.5 | Abil 350 | Dimethicone |
| | 3.0 | Finsolv TN | C12-15 Alkyl Benzoate |
| | 1.0 | Cremophor CO 40 | PEG-40 Hydrogenated |
| | | | Castor Oil |
| | 2.0 | Uvinul ® T150 | Ethylhexyl Triazone |
| B | to 100 | Water | Water |
| | 5.0 | Propylene glycol | Propylene Glycol |
| C | 5.0 | Ethanol | Alcohol |
| | 0.5 | Cremophor A 25 | Ceteareth-25 |
| D | 2.0 | Sepigel 305 | Polyacrylamide, C13-14 |
| | | | Isoparaffin, Laureth-7 |
| E | 1.0 | Euxyl K300 | Phenoxyethanol and |
| | | | Methylparaben and |
| | | | Butylparaben and |
| | | | Ethylparaben and |
| | | | Propylparaben and |
| | | | Isobutylparaben |

### Preparation:

The components of phase A are heated to 80° C.
The components of phase B are dispersed at room temperature with stirring and added to phase A.
The components of phase C are homogenised and added to the mixture of phases A and B and homogenised again.
The components of phases D and E are added to the mixture of phases A+B+C, homogenised again and subsequently cooled to room temperature.
Viscosity: 3450 mPas
pH: 6.5
SPF in vivo COLIPA 6.7

### Formulation 3:

| % by wt. | Raw materials | INCI |
|---|---|---|
| Phase A | | |
| 1.0 | Cremophor A 6 | Ceteareth-6, Stearyl Alcohol |
| 1.0 | Cremophor A 25 | Ceteareth-25 |
| 12.0 | Tegin G | Glycol Stearate SE |
| 10.0 | Miglyol 812 | Caprylic/Capric Triglyceride |
| 10.0 | Witconol APM | PPG-3 Myristyl Ether |
| 1.0 | Cetiol SB 45 | Butyrosperum Parkii (Shea |
| | | Butter) |
| 2.0 | Uvinul T150 | Ethylhexyl Triazone |
| Phase B | | |
| 3.0 | Glycerin 87% | Glycerin |
| 1.5 | Triethanolamine Care | Triethanolamine |
| 3.0 | Uvinul MS 40 | Benzophenone-4 |
| to 100 | Water dem. | Aqua dem. |
| Phase C | | |
| 0.5 | Euxyl K300 | Phenoxyethanol, Methylparaben, |
| | | Butylparaben, Ethylparaben, |
| | | Propylparaben, Isobutylparaben |

### Preparation:

The components of phases A and B are each heated to 80° C. separately from one another, subsequently combined and briefly homogenised.
The mixture is cooled to 40° C. and homogenised again.
The component of phase C is added to the mixture of phases A and B, and everything is homogenised together.
Viscosity (Brookfield SP6): 3350 mPas
pH: 7.0
SPF in vivo COLIPA 6.6

### Formulation 4:

| % by wt. | Raw materials | INCI |
|---|---|---|
| Phase A | | |
| 1.0 | Cremophor A 6 | Ceteareth-6, Stearyl Alcohol |
| 1.0 | Cremophor A 25 | Ceteareth-25 |
| 12.0 | Tegin G | Glycol Stearate SE |
| 10.0 | Miglyol 812 | Caprylic/Capric Triglyceride |
| 10.0 | Witconol APM | PPG-3 Myristyl Ether |
| 1.0 | Cetiol SB 45 | Butyrosperum Parkii (Shea |
| | | Butter) |
| Phase B | | |
| 3.0 | Glycerin 87% | Glycerin |
| 1.5 | Triethanolamine Care | Triethanolamine |
| 3.0 | Uvinul MS 40 | Benzophenone-4 |
| to 100 | Water dem. | Aqua dem. |
| Phase C | | |
| 2.0 | UV filter capsule | Aqua (water), Ethylhexyl |
| | (16.6% of Ethylhexyl | Triazone, Dimethyl |
| | Triazone in Spectrasolv | Capramide, Silica, PVP, |
| | DMDA) corresponds to | Chlorphenesin |
| | 2% of Uvinul T 150 | |
| Phase D | | |
| 0.5 | Euxyl K300 | Phenoxyethanol, |
| | | Methylparaben, Butylparaben, |
| | | Ethylparaben, Propylparaben, |
| | | Isobutylparaben |

### Preparation:

The components of phases A and B are each heated to 80° C. separately from one another, subsequently combined and briefly homogenised.
The components of phase C are added to the mixture of phases A and B, and everything is homogenised together.
The mixture is cooled to 40° C. and homogenised again.
The components of phase D are added to the mixture of phases A+B+C, and everything is homogenised together.
Viscosity (Brookfield SP6): 7450 mPas
pH: 7.3
SPF in vivo COLIPA 6.4.
Formulation 5: O/W cream

| % | Ingredient | Batch (400 g) INCI |
|---|---|---|
| Phase A | | |
| 1.8 | Stearic acid | Stearic Acid |
| 1.8 | Sodium hydroxide 97% | Sodium Hydroxide |
| 10.0 | Water dem. | Aqua dem. |
| Phase B | | |
| 1.0 | Uvinul MC80 | Ethylhexyl Methoxycinnamate |
| 1.0 | Cetiol OE | Dicaprylyl Ether |
| 1.0 | Finsolv TN | C12-15 Alkyl Benzoate |
| 1.0 | Cetiol SN | Cetearyl Isononanoate |
| 1.0 | Abil K 4 | Cyclomethicone |
| 0.5 | Lanette O | Cetearyl Alcohol |
| 1.0 | Uvinul T150 | Ethylhexyl Triazone |
| 3.5 | Glycerol monostearate | Glyceryl Stearate |
| 1.0 | Softisan 100 | Hydrogenated Coco-Glycerides |
| 2.5 | Uvinul A Plus Granular | Diethylamino Hydroxybenzoyl |
| | | Hexyl Benzoate |
| Phase C | | |
| 1.0 | UV filter capsule | Aqua (water), Ethylhexyl Triazone, |
| | (16.6% of Ethylhexyl | Dimethyl Capramide, Silica, PVP, |
| | Triazone in Spectrasolv | Chlorphenesin |
| | DMDA) | |
| 5.0 | 1,2-Propylene glycol Care | Propylene Glycol |
| 3.0 | Glycerin 87% | Glycerin |
| 1.0 | Carbopol 934 | Carbomer |
| 0.3 | Disodium EDTA | Disodium EDTA |
| to 100 | Water dem. | Aqua dem. |
| Phase D | | |
| 1.9 | Citric acid | Citric Acid |
| Phase E | | |
| 0.5 | Vitamin E acetate | Tocopheryl Acetate |
| 4.0 | Ethanol 96% | Alcohol |
| 1.0 | Euxyl K 300 | Phenoxyethanol and |
| | | Methylparaben and Butylparaben |
| | | and Ethylparaben and |
| | | Propylparaben and |
| | | Isobutylparaben |

### Preparation:

Saponification of phase A for 1 h at 80° C.
Melting and mixing of phase B at 80° C.
Mixing of the components of phase C and heating to 80° C. (without capsules).
Admixing of the capsules according to the invention with phase C and homogenising.
Admixing of phases A+B with C and homogenising.
Addition of phase D.
Cooling to room temperature with stirring and addition of phase E with subsequent homogenisation.
SPF in vivo COLIPA 12

### (UV)APF 4

Formulation 6: O/W cream

| % | Ingredient | Batch (400 g) INCI |
|---|---|---|
| Phase A | | |
| 1.8 | Stearic acid | Stearic Acid |
| 1.8 | Sodium hydroxide 97% | Sodium Hydroxide |
| 10.0 | Water dem. | Aqua dem. |
| Phase B | | |
| 1.0 | Uvinul MC80 | Ethylhexyl Methoxycinnamate |
| 1.0 | Cetiol OE | Dicaprylyl Ether |
| 1.0 | Finsolv TN | C12-15 Alkyl Benzoate |
| 1.0 | Cetiol SN | Cetearyl Isononanoate |
| 1.0 | Abil K 4 | Cyclomethicone |
| 0.5 | Lanette O | Cetearyl Alcohol |
| 1.0 | Uvinul T150 | Ethylhexyl Triazone |
| 3.5 | Glycerol monostearate | Glyceryl Stearate |
| 1.0 | Softisan 100 | Hydrogenated Coco-Glycerides |
| 2.5 | Uvinul A Plus Granular | Diethylamino Hydroxybenzoyl |
| | | Hexyl Benzoate |
| Phase C | | |
| 1.0 | Eusolex 232 | Phenylbenzimidazole Sulfonic |
| | | Acid |
| 1.0 | Sodium hydroxide 97% | Sodium Hydroxide |
| 5.0 | 1,2-Propylene glycol Care | Propylene Glycol |
| 3.0 | Glycerin 87% | Glycerin |
| 1.0 | Carbopol 934 | Carbomer |
| 0.3 | Disodium EDTA | Disodium EDTA |
| to 100 | Water dem. | Aqua dem. |
| Phase D | | |
| 2.6 | Citric acid | Citric Acid |
| Phase E | | |
| 0.5 | Vitamin E acetate | Tocopheryl Acetate |
| 4.0 | Ethanol 96% | Alcohol |
| 1.0 | Euxyl K 300 | Phenoxyethanol and |
| | | Methylparaben and Butylparaben |
| | | and Ethylparaben and |
| | | Propylparaben and |
| | | Isobutylparaben |

### Preparation:

Saponification of phase A for 1 h at 80° C.
Melting and mixing of phase B at 80° C.
Mixing of the components of phase C and heating to 80° C.
Admixing of phases A+B with C and homogenising.
Addition of phase D.
Cooling to room temperature with stirring and addition of phase E with subsequent homogenisation.
SPF in vivo COLIPA 8

### (UV)APF 3.8

Formulation 7: O/W emulsion

| % | Ingredient | INCI |
|---|---|---|
| Phase A | | |
| 1.0 | Cremophor A 6 | Ceteareth-6, Stearyl Alcohol |
| 1.0 | Cremophor A 25 | Ceteareth-25 |
| 12.0 | Tegin | Glyceryl Stearate SE |
| 10.0 | Miglyol 812 | Caprylic/Capric Triglyceride |
| 10.0 | Witconol APM | PPG-3 Myristyl Ether |
| 1.0 | Cetiol SB 45 | Butrosperum Parkii (Shea Butter) |
| 2.0 | Uvinul T150 | Ethylhexyl Triazone |
| Phase B | | |
| 3.0 | Glycerin 87% | Glycerin |
| 1.5 | Triethanolamine Care | Triethanolamine |
| 3.0 | Uvinul MS 40 | Benzophenone-4 |
| to 100 | Water dem. | Aqua dem. |
| Phase C | | |
| 0.5 | Euxyl K300 | Phenoxyethanol, Methylparaben, |
| | | Butylparaben, Ethylparaben, |
| | | Propylparaben, Isobutylparaben |

### Preparation:

Heating of phases A and B separately to 80° C.
Add phase B to phase A with stirring and briefly homogenise.
Addition of phase C to combined phases A+B and homogenisation.
Cooling to 40° C. and homogenisation.
Addition of phase C and homogenisation.
Viscosity (Brookfield SP6): 3350 mPas
pH value: 7.0
SPF in vivo COLIPA 6
Formulation 8: O/W emulsion

| % | Ingredient | INCI |
|---|---|---|
| Phase A | | |
| 1.0 | Cremophor A 6 | Ceteareth-6, Stearyl Alcohol |
| 1.0 | Cremophor A 25 | Ceteareth-25 |
| 12.0 | Tegin G | Glycol Stearate SE |
| 10.0 | Miglyol 812 | Caprylic/Capric Triglyceride |
| 10.0 | Witconol APM | PPG-3 Myristyl Ether |
| 1.0 | Cetiol SB 45 | Butyrosperum Parkii (Shea |
| | | Butter) |
| Phase B | | |
| 3.0 | Glycerin 87% | Glycerin |
| 1.5 | Triethanolamine Care | Triethanolamine |
| 3.0 | Uvinul MS 40 | Benzophenone-4 |
| to 100 | Water dem. | Aqua dem. |
| 2.0 | UV filter capsule | Aqua (water), Ethylhexyl Triazone, |
| | (16.6% of Ethylhexyl | Dimethyl Capramide, Silica, PVP, |
| | Triazone in Spectrasolv | Chlorphenesin |
| | DMDA) | |
| Phase C | | |
| 0.5 | Euxyl K300 | Phenoxyethanol, Methylparaben, |
| | | Butylparaben, Ethylparaben, |
| | | Propylparaben, Isobutylparaben |

### Preparation:

Heat phase A and phase B to 80° C. each separately from one another.
Addition of phase B to phase A with stirring and brief homogenisation.
Cool to 40° C. with stirring, and add phase C and homogenise.
Viscosity (Brookfield SP6): 7450 mPas
pH value: 7.3
SPF in vivo COLIPA 6

### Formulation 9:

| % | Ingredient | INCI |
|---|---|---|
| Phase A | | |
| 10.0 | Miglyol 812 | Caprylic/Capric Triglycerides |
| 1.5 | Abil350 | Dimethicone |
| 3.0 | Finsolv TN | C12.15 alkyl Benzoate |
| 1.0 | Cremophor CO 40 | PEG-40 Hydrogenated Castor Oil |
| Phase B | | |
| to 100 | Water | Water |
| 2.0 | UV filter capsule (16.6% | Aqua (water), Ethylhexyl Triazone, |
| | of Ethylhexyl Triazone I | Dimethyl Capramide, Silica, PVP, |
| | Spectrasolv DMDA) | Chlorphenesin |
| 5.0 | Propylene glycol | Propylene Glycol |
| Phase C | | |
| 5.0 | Ethanol | Alcohol |
| 0.5 | Cremophor A 25 | Ceteareth-25 |
| Phase D | | |
| 2.0 | Sepigel 305 | Polyacrylamide, C13-14 Isoparaffin, |
| | | Laureth-7 |
| Phase E | | |
| 1.0 | Euxyl K300 | Phenoxyethanol and Methylparaben |
| | | and Butylparaben and Ethylparaben |
| | | and Propylparaben and |
| | | Isobutylparaben |

### Preparation:

Heating of phase A to 80° C.
Dispersion of phase B at room temperature with stirring.
Homogenisation of phase B in phase A.
Addition of phase C to phases A+B and homogenisation.
After addition of phases D and E, homogenise again.
SPF in vitro 6.

### Formulation 10:

| % | Ingredient | INCI |
|---|---|---|
| Phase A | | |
| 10.0 | Miglyol 812 | Caprylic/Capric Triglycerides |
| 1.5 | Abil350 | Dimethicone |
| 3.0 | Finsolv TN | C12.15 Alkyl Benzoate |
| 1.0 | Cremophor CO 40 | PEG-40 Hydrogenated Castor Oil |
| 2.0 | Uvinul T150 | Ethylhexyl Triazone |
| Phase B | | |
| to 100 | Water | Water |
| 5.0 | Propylene glycol | Propylene Glycol |
| Phase C | | |
| 5.0 | Ethanol | Alcohol |
| 0.5 | Cremophor A 25 | Ceteareth-25 |
| Phase D | | |
| 2.0 | Sepigel 305 | Polyacrylamide, C13-14 Isoparaffin, |
| | | Laureth-7 |
| Phase E | | |
| 1.0 | Euxyl K300 | Phenoxyethanol and Methylparaben |
| | | and Butylparaben and Ethylparaben |
| | | and Propylparaben and |
| | | Isobutylparaben |

### Preparation:

Heating of phase A to 80° C.
Dispersion of phase B at room temperature with stirring.
Homogenisation of phase B in phase A.
Addition of phase C to phases A+B and homogenisation.
After addition of phases D and E, homogenise again.
SPF in vitro 6

### Formulation 11:

| % | Ingredient | Batch (400 g) INCI |
|---|---|---|
| Phase A | | |
| 3.0 | Uvinul T 150 | Ethylhexyl Triazone |
| 10.0 | Uvinul A-Plus Granular | Diethylamino Hydroxybenzoyl |
| | | Hexyl Benzoate |
| 3.0 | Tinosorb S | Bis-Ethylhexyloxyphenol |
| | | Methoxyphenyl Triazine |
| 1.0 | Cutina GMS | Glyceryl Stearate |
| 2.0 | Axol C 62 | Glyceryl Stearate Citrate |
| 7.0 | Finsolv TN | C12-15 Alkyl Benzoate |
| 7.0 | Cetiol B | Dibutyl Adipate |
| 4.0 | Cosmacol ETI | Di-C12-13 Alkyl Tartrate |
| 1.0 | Lanette O | Cetearyl Alcohol |
| 2.0 | Unimer U-6 | Triacontanyl PVP |
| 3.0 | Luvitol Lite | Hydrogenated Polyisobutene |
| Phase B | | |
| 6.0 | T-Lite SF-S | Titanium Dioxide and Hydrated |
| | | Silica and |
| | | Dimethicone/Methicone |
| | | Copolymer and Aluminum |
| | | Hydroxide |
| Phase C | | |
| 2.0 | Glycerin 86% | Glycerin |
| 2.0 | Panthenol 50 P | Panthenol |
| 0.2 | Edeta BD | Disodium EDTA |
| to 100 | Water, demin. | Water |
| 0.15 | Keltrol [E] | Xanthan Gum |
| 0.15 | Polysurf 67 CS | Cetyl Hydroxyethylcellulose |
| 4.8 | Tris Amino Ultra PC(20% in | Tromethamine |
| | water) | |
| 2.0 | Eusolex 232 | Phenylbenzimidazole Sulfonic |
| | | Acid |
| Phase D | | |
| 0.5 | Vitamin E acetate | Tocopheryl Acetate |
| 1.0 | Euxyl K 300 | Phenoxyethanol and |
| | | Methylparaben and |
| | | Butylparaben and Ethylparaben |
| | | and Propylparaben and |
| | | Isobutylparaben |

### Preparation:

Heating of phase A to 80° C.
Addition of phase B to the molten phase A and homogenisation.
Homogenisation of phase C until clear.
Addition of phase C to phases A+B and homogenisation.
Cooling to 40° C. with stirring.
Addition of phase D, brief homogenisation and cooling to room temperature with stirring.
SPF in vivo COLIPA 30

### Formulation 12:

| % | Ingredient | INCI |
|---|---|---|
| Phase A | | |
| 3.0 | Uvinul T 150 | Ethylhexyl Triazone |
| 10.0 | Uvinul A-Plus Granular | Diethylamino Hydroxybenzoyl Hexyl |
| | | Benzoate |
| 3.0 | Tinosorb S | Bis-Ethylhexyloxyphenol |
| | | Methoxyphenyl Triazine |
| 1.0 | Cutina GMS | Glyceryl Stearate |
| 2.0 | Axol C 62 | Glyceryl Stearate Citrate |
| 7.0 | Finsolv TN | C12-15 Alkyl Benzoate |
| 7.0 | Cetiol B | Dibutyl Adipate |
| 4.0 | Cosmacol ETI | Di-C12-13 Alkyl Tartrate |
| 1.0 | Lanette O | Cetearyl Alcohol |
| 2.0 | Unimer U-6 | Triacontanyl PVP |
| 3.0 | Luvitol Lite | Hydrogenated Polyisobutene |
| Phase B | | |
| 6.0 | T-Lite SF-S | Titanium Dioxide and Hydrated |
| | | Silica and Dimethicone/Methicone |
| | | Copolymer and Aluminum Hydroxide |
| Phase C | | |
| 2.0 | Glycerin 86% | Glycerin |
| 2.0 | Panthenol 50 P | Panthenol |
| 0.2 | Edeta BD | Disodium EDTA |
| to 100 | Water, demin. | Water |
| 0.15 | Keltrol [E] | Xanthan Gum |
| 0.15 | Polysurf 67 CS | Cetyl Hydroxyethylcellulose |
| 4.8 | Tris Amino Ultra | Tromethamine |
| | PC(20% in water) | |
| 12.05 | UV filter capsule (16.6% | Aqua (water), Ethylhexyl Triazone, |
| | of Ethylhexyl Triazone in | Dimethyl Capramide, Silica, PVP, |
| | Spectrasolv DMDA) | Chlorphenesin |
| Phase D | | |
| 0.5 | Vitamin E acetate | Tocopheryl Acetate |
| 1.0 | Euxyl K 300 | Phenoxyethanol and Methylparaben |
| | | and Butylparaben and Ethylparaben |
| | | and Propylparaben and |
| | | Isobutylparaben |

### Preparation:

Heating of phase A to 80° C.
Addition of phase B to the molten phase A and homogenisation.
Homogenisation of phase C until clear. Addition of phase C to phases A+B and homogenisation.
Cooling to 40° C. with stirring.
Addition of phase D, brief homogenisation and cooling to room temperature with stirring.
SPF in vivo COLIPA 33
(UV)APF in vitro 11.

1. UV filter capsule comprising
a polymeric shell and
a) at least one low-solubility organic UV filter and
b) an emollient which is capable of dissolving more than 40% by weight of the low-solubility organic UV filter at room temperature.

2. UV filter capsule according to claim 1, characterised in that the emollient mentioned under b) corresponds to a compound of the formula I 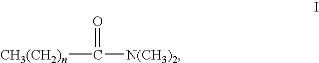
where n corresponds to an integer from 2 to 12.

3. UV filter capsule according to claim 1, characterised in that the at least one low-solubility organic UV filter mentioned under a) is a triazine derivative, diarylbutadiene derivative, hydroxybenzophenone derivative and/or methylenebisbenzotriazolyltetramethylbutylphenol derivative.

4. UV filter capsule according to claim 1, where the at least one low-solubility organic UV filter mentioned under a) is a triazine derivative selected from the group comprising 2,4,6-tris-[anilino-(p-carbo-2′-ethyl-1′-hexyloxy)]-1,3,5-triazine, dioctylbutamidotriazone, bisethylhexyloxyphenolmethoxyphenyltriazine, 2,4,6-tris(diethyl-4′-aminobenzal malonate)-s-triazine, 2,4,6-tris(dimethyl-4′-aminobenzal malonate)-s-triazine, 2,4,6-tris(diisopropyl-4-aminobenzal malonate)-s-triazine, 2,4,6-tris[3′-benzotriazol-2-yl)-2′-hydroxy-5′-methyl)-phenylamino]-s-triazine and 2,4,6-tris[3′-benzotriazol-2-yl)-2′-hydroxy-5′-tert-octyl)phenylamino]-s-triazine.

5. UV filter capsule according to claim 1, where the at least one low-solubility organic UV filter mentioned under a) is a triazine derivative selected from 2,4,6-tris[anilino-(p-carbo-2′-ethyl-1′-hexyloxy)]-1,3,5-triazine, dioctylbutamidotriazone or bisethylhexyloxyphenolmethoxyphenyltriazine.

6. UV filter capsule according to claim 1, where the at least one low-solubility organic UV filter mentioned under a) is a diarylbutadiene derivative, and the diarylbutadiene conforms to the formula II 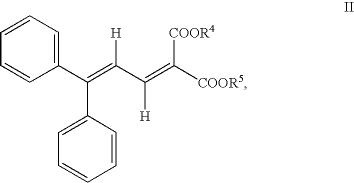
where R⁴ and R⁵, independently of one another, denote hydrogen, C₁-C₂₀-alkyl, C₃-C₁₀-cycloalkyl or C₃-C₁₀-cycloalkenyl.

7. UV filter capsule according to claim 1, where the at least one low-solubility organic UV filter mentioned under a) is 1,1-dicarboxy(2′,2′-dimethylpropyl)-4,4-diphenylbutadiene.

8. UV filter capsule according to claim 1, where the at least one low-solubility organic UV filter mentioned under a) corresponds to a hydroxybenzophenone of the formula III 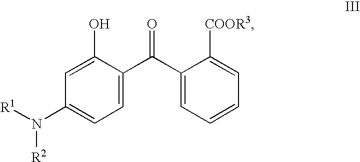
where R¹ and R², independently of one another, denote H, C₁-C₂₀-alkyl, C₃-C₁₀-cycloalkyl or C₃-C₁₀-cycloalkenyl, where the substituents R¹ and R², together with the nitrogen atom to which they are bonded, may form a 5- or 6-membered ring, and R³ denotes C₁-C₂₀-alkyl.

9. UV filter capsule according to claim 1, where the at least one low-solubility organic UV filter mentioned under a) is hexyl 2-(4-diethylamino-2-hydroxybenzoyl)benzoate.

10. UV filter capsule according to claim 1, where the at least one low-solubility organic UV filter mentioned under a) is a methylenebisbenzotriazolyltetramethylbutylphenol derivative.

11. UV filter capsule according to claim 1, where the at least one low-solubility organic UV filter mentioned under a) is 2,2′-methylenebis [6-(2H-benzotriazol-2-yl)-4-(1,1,3,3-tetramethylbutyl)phenol].

12. UV filter capsule according to claim 1, characterised in that the UV filter capsule contains the at least one low-solubility UV filter mentioned under a) and the emollient mentioned under b) in the weight percent ratio 10:90 to 90:10, preferably in the weight percent ratio 30:70 to 70:30.

13. Dispersion comprising UV filter capsules according to claim 1.

14. Dispersion according to claim 13, where the dispersion is aqueous.

15. Dispersion according to claim 13, where the proportion of UV filter capsules is 5 to 80% by weight, based on the total amount of the dispersion.

16. Composition comprising at least one low-solubility organic UV filter and at least one suitable vehicle, characterised in that at least some of the low-solubility organic UV filter is encapsulated in the form of UV filter capsules according to claim 1.

17. (canceled)

18. Process for the preparation of a composition, comprising mixing UV filter capsules according to claim 1 with further ingredients.

19. Process for the production of UV filter capsules according to claim 1, characterised in that
in step a), an oil-in-water emulsion is prepared from a mixture comprising a sol-gel precursor for the production of the polymeric shell, at least one low-solubility UV filter and an emollient which is capable of dissolving more than 40% by weight of the low-solubility organic UV filter at room temperature, in an aqueous solution,
in step b), the emulsion prepared in step a) is mixed to give an aqueous solution having a pH of 2 to 4, and optionally
in step c), reaction products are separated off from the sol-gel precursor, and the UV filter capsules are isolated.

20. A process for the preparation of a composition, comprising mixing a dispersion according to claim 3 with further ingredients.
